(19) United States
(12) Patent Application Publication
(10) Pub. No.: US 2020/0354731 A1
(43) Pub. Date: Nov. 12, 2020

(54) ENDOPLASMIC RETICULUM TARGETING SIGNAL

(71) Applicant: Yeda Research and Development Co. Ltd., Rehovot (IL)

(72) Inventors: Jeffrey E. GERST, Nes Ziona (IL); Lisha Qiu Jin LIM, Rehovot (IL); Yitzhak PILPEL, Rehovot (IL); Tsviya OLENDER, Rehovot (IL); Dvir DAHARY, Rehovot (IL); Osnat COHEN-ZONTAG, Rehovot (IL)

(73) Assignee: Yeda Research and Development Co. Ltd., Rehovot (IL)

(21) Appl. No.: 16/940,440

(22) Filed: Jul. 28, 2020

Related U.S. Application Data
(63) Continuation of application No. PCT/IL2019/050128, filed on Jan. 31, 2019.

(30) Foreign Application Priority Data
Jan. 31, 2018 (IL) 257269

(51) Int. Cl. C12N 15/81; C07K 14/435

(52) U.S. Cl. CPC C12N 15/81; C12N 2810/40; C07K 14/435

(57) ABSTRACT

Isolated polynucleotides comprising a transcriptional unit are disclosed, the transcriptional unit comprising:

(i) a nucleic acid sequence that encodes a secreted protein of interest;

(ii) an endoplasmic reticulum (ER) targeting sequence as set forth in SEQ ID NO: 2, said ER targeting sequence being heterologous to said secreted protein of interest;

(iii) a promoter; and

(iv) a transcription termination site.

### RELATED APPLICATIONS

This application is a Continuation of PCT Patent Application No. PCT/IL2019/050128 having International filing date of Jan. 31, 2019, which claims the benefit of priority of Israel Patent Application No. 257269 filed on Jan. 31, 2018. The contents of the above applications are all incorporated by reference as if fully set forth herein in their entirety.

### SEQUENCE LISTING STATEMENT

The ASCII file, entitled 83080SequenceListing.txt, created on Jul. 28, 2020, comprising 4,231 bytes, submitted concurrently with the filing of this application is incorporated herein by reference. The sequence listing submitted herewith is identical to the sequence listing forming part of the international application.

### FIELD AND BACKGROUND OF THE INVENTION

The present invention, in some embodiments thereof, relates to methods of enhancing expression of recombinant proteins and, more particularly, but not exclusively, to secreted recombinant proteins.

mRNA targeting and localized translation is an important mechanism that provides spatial and temporal control of protein synthesis. The delivery of mRNA to specific subcellular compartments has a major role in the establishment of polarity in various of organisms and cell types, and was shown to be crucial for the proper function of the cell. Interestingly, the localization of mRNA is often governed by cis-acting elements (zipcodes) embedded within the mRNA sequence (Martin and Ephrussi, 2009; Buxbaum et al., 2014). RNA binding proteins (RBPs) recognize such sequences and act together with molecular motors to direct the mRNAs to their destination.

The endoplasmic reticulum (ER) is the site of synthesis of secreted and membrane (SMP; secretome) proteins. According to dogma, mRNAs encoding for SMPs (mSMPs) are delivered to the ER by a distinct translation-dependent mechanism, carried out by the signal recognition particle (SRP) pathway. According to this model, protein translation begins in the cytoplasm and when SMP transcripts undergo translation, a signal peptide present at the amino terminus of their polypeptide emerges from the exit tunnel of translating ribosome and is recognized by the SRP. The SRP is then recruited to its receptor on the ER membrane and translocation of ribosome-mRNA-nascent protein chain complex from the cytoplasm to the ER occurs. There, translating ribosomes interact with the translocon to enable co-translational protein translocation and mRNA anchoring. Thus, the SRP model describes the mSMP as a component with no active role in the ER translocation process.

However, multiple lines of evidence suggest that there are additional pathways for the delivery of mRNAs to the ER. First, loss of the SRP pathway did not result in lethality of yeast and mammalian cells, and also did not have a significant effect upon membrane protein synthesis and global mRNA distribution between the cytoplasm and the ER. Second, genome-wide analyses of the distribution of mRNAs encoding soluble and membrane proteins between cytosolic polysomes and ER-bound polysomes have demonstrated a significant overlap in the composition of the mRNA in the two fractions and showed that cytosolic protein-encoding mRNAs are broadly represented on the ER. This means that mRNAs lacking an encoded signal sequence can localize to the ER. In agreement with these findings, removal of the signal sequence and the inhibition of translation did not disrupt mSMP localization to the ER (Pyhtila et al., 2008; Chen et al., 2011; Kraut-Cohen et al., 2013). Third, subsets of secretome proteins are known to localize to the ER in an SRP-independent pathway. These proteins are thought to translocate into the ER after translation in the cytosol. In a study that utilized a technique for a specific pull-down of ER-bound ribosomes (Jan et al., 2014), it was found that there is no significant difference in the enrichment of mRNAs encoding SRP-dependent proteins in comparison to mRNAs encoding SRP-independent proteins on ER membranes. In addition, a subset of ribosomes managed to reach the ER before the emergence of the signal sequence. A possible explanation for these observations could be that mRNAs reach the ER before the ribosomes in an SRP-independent mechanism. If mRNA targeting to the ER does not begin until signal peptide emergence, membrane-bound ribosome should not be translating the portion of transcript upstream of the signal peptide. However, this is not the case, as translating membrane-bound ribosomes were found to be evenly distributed across the entire transcript (Chartron et al., 2016). This suggests that mRNA is localized to the ER before translation initiation.

Although it has been difficult to identify clear cis-elements within mRNA that direct it to the ER, specific sequence characteristics of mSMPs have been identified. For example, sequence analysis of the region encoding the signal sequence revealed a low usage of adenine to create no-A stretches within this sequence (Palazzo et al., 2007). Additionally, mRNAs encoding membrane proteins have a high degree of uracil enrichment, as well as pyrimidine usage, in comparison to mRNAs encoding cytosolic proteins (Wolfenden et al., 1979; Prilusky and Bibi, 2009; Kraut-Cohen and Gerst, 2010; Polyansky et al., 2013). These findings raise the possibility that the ER localization motif resides in a more diffuse, general fashion in the sequence composition of the mRNA molecule.

### SUMMARY OF THE INVENTION

According to an aspect of the present invention there is provided an isolated polynucleotide comprising a transcriptional unit, wherein the transcriptional unit comprises:

(i) a nucleic acid sequence that encodes a secreted protein of interest;

(ii) an endoplasmic reticulum (ER) targeting sequence as set forth in SEQ ID NO: 2, said ER targeting sequence being heterologous to said secreted protein of interest;

(iii) a promoter; and

(iv) a transcription termination site, wherein the nucleic acid sequence that encodes a protein of interest and the ER targeting sequence are positioned between the promoter and the transcription termination site;

wherein when the ER targeting sequence is comprised in the nucleic acid sequence that encodes the protein of interest, the nucleic acid sequence has been codon optimized to comprise the ER targeting sequence.

According to an aspect of the present invention there is provided a method of generating a protein comprising expressing the protein according the methods described herein and isolating the protein, thereby generating the protein.

According to an aspect of the present invention there is provided an RNA transcribed from the polynucleotide described herein.

According to an aspect of the present invention there is provided a cell comprising the isolated polynucleotide described herein.

According to an aspect of the present invention there is provided an expression construct comprising the polynucleotide described herein.

According to an aspect of the present invention there is provided an expression construct comprising a nucleic acid sequence as set forth in SEQ ID NO: 2 and a cloning site, wherein a position of the cloning site is selected such that upon insertion of a sequence which encodes a protein of interest into the cloning site, following expression in a cell, an mRNA is transcribed which encodes the protein of interest and further comprises a transcription product of the nucleic acid sequence, wherein the SEQ ID NO: 2 is not comprised in a sequence that encodes for a protein.

According to an aspect of the present invention there is provided a method of expressing a protein in a cell, the method comprising introducing into the cell the isolated polynucleotide described herein, thereby expressing the protein.

According to an embodiment, the ER targeting sequence does not comprise nucleotides that encode for the secreted protein of interest.

According to embodiments of the present invention, the ER targeting sequence does not comprise nucleotides that encode for a sequence as set forth in SEQ ID NO: 5.

According to embodiments of the present invention, the ER targeting sequence does not comprise the sequence as set forth in SEQ ID NO: 6.

According to embodiments of the present invention, the ER targeting sequence does not comprise more than 5 consecutive repeats of the sequence TG.

According to embodiments of the present invention, the ER targeting sequence comprises at least 15 consecutive repeats of the sequence NNY, wherein N is any base and Y is a pyrimidine.

According to embodiments of the present invention, the ER targeting sequence does not comprise more than 10 consecutive thymines.

According to embodiments of the present invention, the ER targeting sequence is positioned 3′ to the nucleic acid sequence that encodes a protein of interest.

According to embodiments of the present invention, the ER targeting sequence nucleic acid sequence is positioned 5′ to the nucleic acid sequence that encodes a protein of interest.

According to embodiments of the present invention, the transcriptional unit further encodes a signal peptide sequence.

According to embodiments of the present invention, the signal peptide sequence is heterologous to the protein of interest.

According to embodiments of the present invention, the protein of interest is a human protein.

According to embodiments of the present invention, the protein of interest is selected from the group consisting of an antibody, insulin, interferon, growth hormone, erythropoietin, growth hormone, follicle stimulating hormone, factor VIII, low density lipoprotein receptor (LDLR) alpha galactosidase A and glucocerebrosidase.

According to embodiments of the present invention, the cell is of a species selected from the group consisting of a bacterial species, a fungal species, a plant species, an insect species and a mammalian species.

According to embodiments of the present invention, the cells of a bacterial species comprise E. coli cells.

According to embodiments of the present invention, the cells of a mammalian species comprise Chinese hamster ovary (CHO) cells.

According to embodiments of the present invention, the cells of a fungal species comprise S. cerevisiae cells.

According to embodiments of the present invention, the expression construct further comprises a promoter suitable for expressing the protein of interest in a cell.

According to embodiments of the present invention, the cell is of a species selected from the group consisting of a bacterial species, a fungal species, a plant species, an insect species and a mammalian species.

According to embodiments of the present invention, the cells of a bacterial species comprise E. coli cells.

According to embodiments of the present invention, the cells of a mammalian species comprise Chinese hamster ovary (CHO) cells.

According to embodiments of the present invention, the cells of a fungal species comprise S. cerevisiae cells.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C. Determination of the number of NNY repeats to use as a threshold for SECReTE. (A) Correlation between SECReTE number and transcript length. The total SECReTE score was calculated for each yeast gene (5904 scored) by counting the number of consecutive NNY repeats present in the transcript sequence according to the indicated threshold, and in all three frames. Scatter plots represent the correlation between the SECReTE score and gene length. The SECReTE score does not correlate with gene lengths above a threshold of 10 NNY repeats (SECReTE10). R score represents the Pearson correlation coefficient. (B) SECReTE motifs are more abundant in the mRNAs coding for secretome proteins than for non-secretome proteins. SECReTE presence, according to the indicated threshold, was scored in mRNAs coding for secretome (blue) and non-secreted (gray) proteins. Bars represent the fraction of SECReTE positive transcripts at the indicated threshold. SECReTE abundance is significantly higher in secretome mRNAs. *p≤2.28 E−13. (C) SECReTE10 maximizes the ability to distinguish secretome transcripts. ROC curves were plotted for each of the indicated thresholds. Secretome transcripts were used as the “true positive” set, while non-secretome transcripts were used as the “true negative” set. The AUC (area under the curve) of SECReTE10 was the highest.

FIGS. 2A-C. SECReTE abundance in mSMPs is transmembrane domain (TMD)-independent. (A) SECReTE is abundant in the second position of the codon. SECReTE abundance was calculated for each codon position separately. SECReTE abundance in mSMPs is most significant in the second codon position, but significant differences were also detected in the third position, *p≤9.9 E−10. (B) SECReTE is also highly abundant in the mRNAs encoding soluble secretome proteins. SECReTE10 presence was examined separately for TMD-containing proteins and soluble secreted proteins. A higher fraction of mRNAs coding for soluble secreted proteins (Secretome without TMD; cyan) contains SECReTE in comparison to non-secretome transcripts, either with or without a TMD (Non-secretome with TMD; dark gray, Non-secretome without TMD; light gray). In the third codon position (NNY), the fraction of soluble secreted proteins is even larger than TMD-containing secretome proteins and is significant, *p≤3.03 E−3. (C) SECReTE is abundant at the third position after removal of the TMD sequence. SECReTE10 presence was scored in mRNAs coding for membrane proteins after the encoded TMD was removed. SECReTE10 is significantly more abundant in the third position (NNY) in mRNAs encoding secretome proteins (blue) than non-secretome proteins (gray), even after removal of the TMD sequence. *p=0.01.

FIGS. 3A-D. Cell wall proteins are highly enriched with SECReTE. (A) GO annotation analysis for genes containing SECReTE10. Genes encoding cell wall proteins, as well as membrane proteins, show the highest and most significant enrichment score. (B) GO annotation analysis for genes containing SECReTE15. Genes encoding cell wall proteins are the most enriched with SECReTE. (C) SECReTE10 abundance in different groups of genes. More than 90% of mRNAs encoding proteins annotated to localize to the cell wall contain SECReTE. High SECReTE abundance was also noticed in other secretome groups except tail-anchored (TA) proteins. Mitochondrial mRNAs (Mito) have low SECReTE abundance. Numbers above bars represent the number of genes in each group. (D) MEME analysis of cell wall transcripts. A motif similar to SECReTE was revealed in cell wall transcripts using MEME. Numbers on the x axis indicate base number.

FIGS. 4A-E. SECReTE is found in the human genome. (A) SECReTE10 maximizes the ability to classify secretome genes in human. ROC curves were plotted for each of the indicated thresholds. Secretome genes were used as the true positive set and non-secretome genes as the true negative set. The AUC (area under the curve) of SECReTE10 was the highest. B. SECReTE is highly abundant in the mRNAs of human secretome proteins. SECReTE10 abundance was calculated for each codon position separately. SECReTE abundance in human mSMPs is most significant in the second position of the codon, but highly significant differences were also detected in the third position. *p≤3.73 E−68 C. SECReTE is highly abundant in mRNAs coding for soluble secretome proteins in humans. SECReTE10 presence was examined separately for TMD-containing proteins and soluble secreted proteins. A higher fraction of mRNAs coding for soluble secreted proteins (Secretome without TMD; cyan) contains SECReTE in comparison to non-secretome transcripts, either with or without a TMD (Non-secretome with TMD; dark gray, Non-secretome without TMD; light gray). The fraction of soluble secreted proteins having SECReTE in the third position is larger than that of TMD-containing non-secretome proteins (NNY) and is significant. Numbers above bars represent the number of genes in each group. * p≤3.49 E−12. (D) SECReTE10 abundance in different groups of genes. High SECReTE abundance was observed for other secretome protein groups, except tail-anchored (TA) proteins. Mitochondrial mRNAs (Mito) have low SECReTE abundance. Numbers above bars represent the number of genes in each group. (E) SECReTE10 abundance in B. subtilis. SECReTE10 abundance was scored and was observed to be higher in mRNA coding for genes encoding secretome proteins (i.e. SS&TMD, TMD, and SS) as compared to those encoding non-secretome (Non-Sec) proteins. Numbers under bars represent the number of genes in each group.

FIGS. 5A-F. The levels of secretion of endogenous and exogenous proteins are affected by SECReTE strength. (A) SECReTE enhances the ability to grow on sucrose. The ability of WT, suc2Δ, SUC2(+)SECReTE and SUC2(−)SECReTE yeast to grow on sucrose was examined by drop-test. Cells were grown to mid-log on glucose-containing YPD medium, prior to serial dilution and plating onto sucrose-containing synthetic medium or YPD. Cells were grown for 2 days prior to photodocumentation. The SUC2(−)SECReTE mutant exhibited reduced growth than WT cells, while SUC2(+)SECReTE cells exhibited better growth. suc2Δ cells were unable to grow on sucrose-containing medium. (B) SECReTE enhances invertase secretion. The indicated strains from A were subjected to the invertase secretion assay. Both internal and secreted invertase activity was measured in units after glucose de-repression. Both activities were reduced in SUC2(−)SECReTE cells and elevated in SUC2(+)SECReTE cells. Error bars represent the standard deviation from three experimental repeats. *p<0.05. (C) SECReTE enhances the ability to grow on calcofluor white. The ability of WT, hsp150Δ, HSP150(+)SECReTE and HSP150(−)SECReTE cells to grow on CFW was examined by drop-test. Cells were grown to mid-log on YPD, prior to serial dilution and plating on YPD alone or YPD plates containing CFW, and incubated at 30° C. Cells were grown for 2 days prior to photodocumentation. The HSP150(−)SECReTE mutant exhibited hypersensitivity in comparison to WT cells, while HSP150(+)SECReTE cells were less sensitive. hsp150Δ cells grew poorly on medium containing CFW. (D) SECReTE enhances Hsp150 secretion. The indicated strains from C were subjected to the Hsp150 secretion assay. Cells were grown to mid-log phase at 37° C. for 4 hrs and examination in cell lysates (internal) or medium (external) by Western analysis using anti-Hsp150 antibodies. External Hsp150 was decreased in HSP150(−)SECReTE cells in comparison to WT, while it was increased in the HSP150(+)SECReTE strain. Internal Hsp150 was decreased in HSP150(−)SECReTE cells and also slightly in HSP150(+)ERTM cells, in comparison with WT cells. No internal nor external Hsp150 was detected in the lysate or medium derived from hsp150Δ cells, respectively. Band intensity was quantified using ImageJ and presented in the histogram below. The graphs represent the ratio of the intensity of all samples relative to that of WT. (E) SECReTE enhances the ability to grow on hygromycin B. The ability of WT, ccw12Δ, and CCW12(−)SECReTE cells to grow on HB was examined by drop-test. Cells were grown to mid-log on glucose-containing YPD medium, prior to serial dilution and plating onto HB-containing YPD or YPD alone. Cells were grown for 2 days prior to photodocumentation. The CCW12(−)SECReTE strain was more sensitive to HB stress in comparison to WT cells. ccw12Δ cells were unable to grow on medium containing HB. (F) SECReTE enhances secretion of an exogenous protein, SSGAS1-GFP. Yeast expressing SSGAS1-GFP3′UTRGAS1(+)SECReTE, SSGAS1-GFP, SSKAR2-GFP, GFP, and SSGAS1-LacZ from plasmids were grown to mid-log phase on synthetic medium containing 2% raffinose and shifted to 3% galactose-containing medium for 4 hrs. Proteins expressed from the different strains were TCA precipitated from the medium and the precipitates resolved by SDS-PAGE. GFP was detected with an anti-GFP antibody, while Hsp150 was detected with an anti-Hsp150 antibody and was used as a loading control. Band intensity was quantified using ImageJ; intensity was scored relative to SSGAS1-GFP secretion. Addition of the GAS1 3′UTR mutated to contain SECReTE improved the secretion of SS-Gas1 and was comparable to that of SSKAR2-GFP. GFP lacking a SS was not secreted and SSGAS1-LacZ was used as a negative control.

FIGS. 6A-B. SECReTE enhances SUC2 mRNA localization to the ER. (A) Visualization of endogenously expressed SUC2(+)SECReTE and SUC2(−)SECReTE mRNAs using smFISH. Yeast endogenously expressing WT SUC2, SUC2(+)SECReTE, or SUC2(−)SECReTE and Sec63-GFP from a plasmid were grown to mid-log phase on SC medium containing 2% glucose prior to shifting cells to low glucose-containing medium (0.05% glucose) to induce SUC2 expression. Cells were processed for smFISH labeling using non-overlapping, TAMRA-labeled, FISH probes complementary to SUC2. B. Quantification of SUC2(+)SECReTE and SUC2(−)SECReTE mRNA localization to the ER. The percentage of granules that are co-localized, not co-localized, or adjacent to Sec63-GFP labeled ER was scored in each cell. The histogram shows the average score for at least ˜60 cells and ˜250 SUC2 granules for each strain, *p=0.019.

FIGS. 7A-B. Identification of potential SECReTE-binding proteins. (A) Identification of SECReTE10-containing transcripts in RNA-binding protein pulldown studies. The number and fraction of SECReTE10-containing mRNAs from the total mRNAs bound to the indicated RBPs is shown. The microarray analysis data used to generate the histogram was published in (Colomina et al., 2008; Hasegawa et al., 2008; Hogan et al., 2008). (B) Identification of potential SECReTE-binding partners. WT cells and either WT or HSP150(+)SECReTE cells deleted for genes encoding the indicated RBPs (e.g. Whi3, and Khd1) were grown to mid-log phase on YPD at 30° C., prior to serial dilution and plating onto either solid YPD medium or YPD containing CFW. Yeast were grown 2 days prior to photodocumentation.

FIG. 8. SECReTE plays an active role in protein secretion. SECReTE-containing transcripts (1) bind SBPs (2) and induce mRNA targeting to the ER (3) and/or confer mRNA stabilization (4). Targeting to the ER may provide spatial regulation and mRNA stabilization (5), leading to subsequent increases in protein production (6) and secretion (7).

FIGS. 9A-D. SECReTE abundance is not dependent on codon composition. Permutation analysis was conducted to evaluate the dependency of SECReTE on codon usage. To do that, codon composition was kept and sequences were randomly reshuffled 1000 times. The Z-score was calculated for each gene to assess the probability of the SECReTE10 to appear randomly (for Z-score calculation, see Materials and Methods). The higher the Z-score the less likely it is for SECReTE to appear randomly. (A) SECReTE enrichment in secretome-encoding mRNAs is independent of codon composition. Distribution plots of Z-scores show higher values for mRNAs encoding secretome proteins than for non-secretome proteins. (B) SECReTE enrichment in mRNAs encoding both soluble and membranal secretome transcripts is independent of codon composition. Distribution plots of Z-scores show higher values for mRNAs encoding secretome proteins (mSMPs; either with or without a TMD) than for non-secretome proteins (i.e. with or without a TMD). (C) SECReTE enrichment in the second and third position of the codon is independent of codon usage. The fraction of significant Z-scores (i. e. ≥1.96) is larger for mRNAs encoding secretome proteins than for non-secretome proteins. (D) SECReTE enrichment in the second and third position of the codon is independent of both codon usage and TMD presence. The fraction of significant Z-scores (i. e. ≥1.96) is larger for mRNAs encoding secretome proteins than for non-secretome proteins, either with or without a TMD.

FIGS. 10A-C. Illustration of SECReTE and SECReTE mutations in SUC2, HSP150, and CCW12. Graphs compare the number of NNY repeats found along the length of the gene either with (lower schematics) or without using a threshold of 10 consecutive NNY repeats (upper schematics) in the native and mutant SECReTE genes. (A) SUC2. (B) HSP150. (C) CCW12.

FIGS. 11A-C. Mutations in SECReTE do not necessarily affect mRNA levels. mRNA levels of native or mutant SUC2, CCW12, and HSP150 in the indicated strains were quantified by qRT-PCR. Fold-change was calculated relative to WT levels. (A) SUC2 mRNA levels are altered by SECReTE mutation. Cells were grown to mid-log phase on SC medium containing 2% glucose at 30° C. prior to shifting cells to low glucose medium for 1.5 hrs. After harvesting and RNA extraction, primers used for amplifying the long transcript of SUC2, which encodes the secreted protein. Primers for actin were used for normalization. SUC2(−)SECReTE cells exhibited lower SUC2 mRNA levels than WT, while SUC2(+)SECReTE cells yielded higher levels. Error bars represent the standard deviation of three biological repeats. (B) CCW12 mRNA levels are not altered by SECReTE mutation. Cells were grown to mid-log phase on YPD medium at 30° C. prior to harvesting and RNA extraction. Primers used for amplifying UBC6 were used for normalization. CCW12 mRNA levels were not significantly changed as a result of SECReTE alterations. (C) HSP150 mRNA levels are not altered by SECReTE mutation. Yeast strains were grown to mid-log phase at either 26° C. or 37° C. on YPD medium prior to harvesting and RNA extraction. UBC6 was used for normalization. HSP150 mRNA levels were not significantly changed as a result of SECReTE alterations.

FIGS. 12A-B. Identification of potential SECReTE-binding proteins. WT cells and either WT or HSP150(+)SECReTE cells deleted for genes encoding the indicated RBPs [e.g. Puf2, She2 (A) and Puf1(B)] were grown to mid-log phase on YPD at 30° C., prior to serial dilution and plating onto either solid YPD medium or YPD containing CFW. Yeast were grown 2 days prior to photo documentation.

FIG. 13 is an exemplary sequence that may be used for expression of GFP—(SEQ ID NO: 4).

### DESCRIPTION OF SPECIFIC EMBODIMENTS OF THE INVENTION

The present invention, in some embodiments thereof, relates to methods of enhancing expression of recombinant proteins and, more particularly, but not exclusively, to secreted recombinant proteins.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The sorting of proteins to their proper destination is crucial for cellular organization and normal function. While the information for protein localization can reside within the protein sequence (e.g. protein targeting sequences), the spatial localization of an mRNA may also be important for correct protein intracellular targeting.

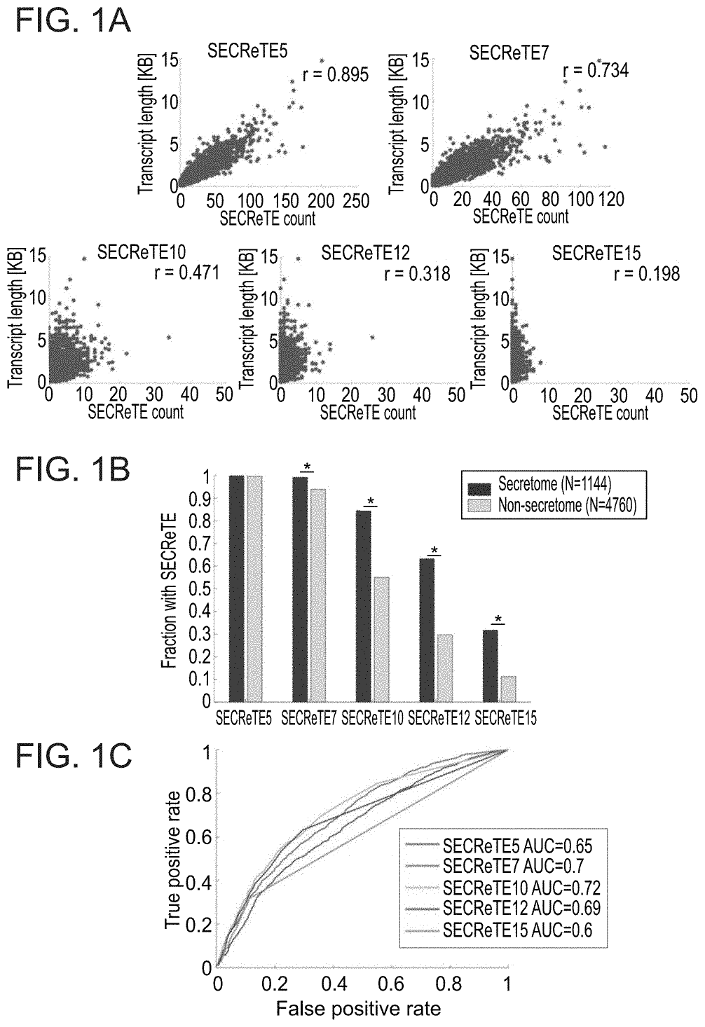

The present inventors have now identified features that characterize all secreted and membrane proteins (SMPs), and discovered a repetitive motif consisting of >10 consecutive NNY repeats. This motif, referred to herein as “SECReTE”, is not restricted to transcripts coding for transmembrane domain (TMD)-containing proteins, but can be found in higher abundance in all secretome transcripts, from prokaryotes (e.g. B. subtilis) to yeast (S. cerevisiae and S. pombe) to humans (FIGS. 1A-C and 4A-E).

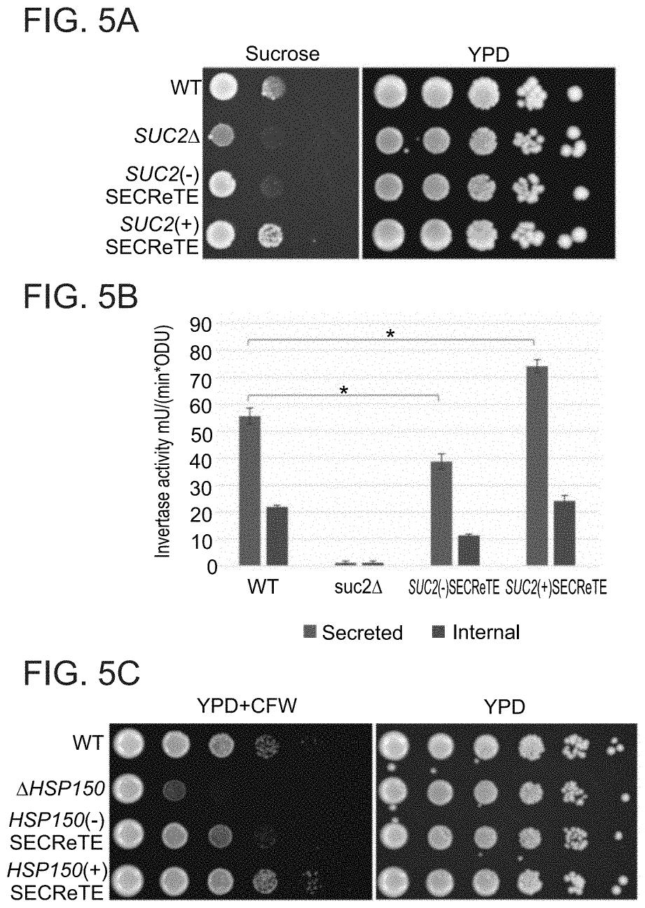

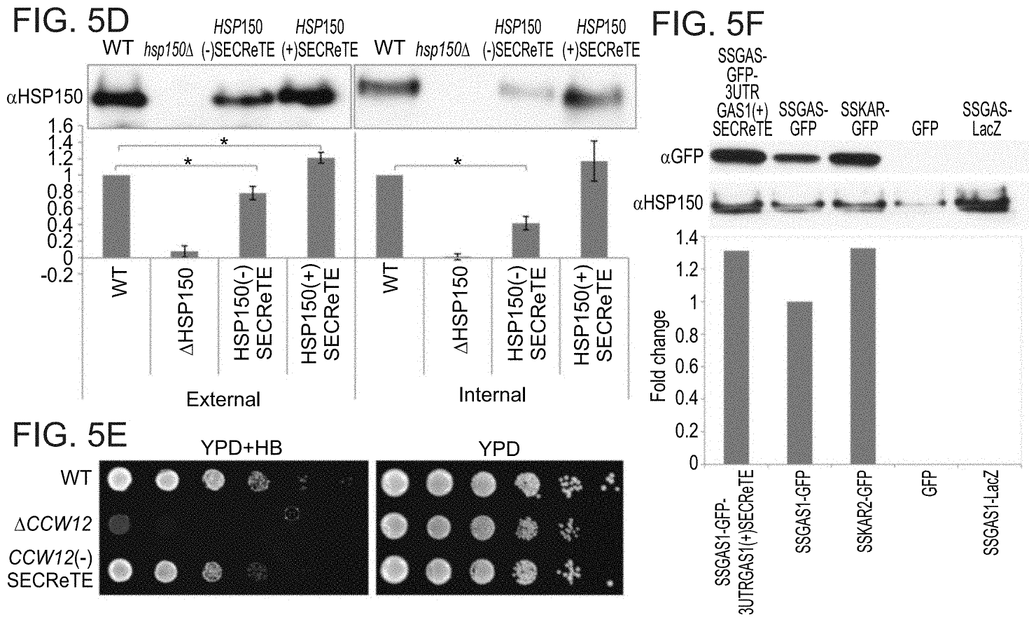

The physiological relevance of SECReTE was explored by altering its enrichment in three mRNAs encoding SMPs: SUC2, HSP150, and CCW12 (FIG. 5A-F). Although the amino acid sequences were not altered by mutation, the functionality of these genes was. SUC2 SECReTE mutant exhibited altered growth rates on sucrose-containing medium in comparison to WT cells, i.e. reduced growth when motif strength was decreased and better growth when motif strength was elevated (FIG. 5A). This result corresponded with either a decrease or increase in both invertase synthesis and secretion, respectively (FIG. 5A, B). HSP150 SECReTE mutants also behaved differently, i.e. HSP150(−)SECReTE cells exhibited higher sensitivity to CFW in comparison to WT cells, while HSP150(+)SECReTE cells were more resistant (FIG. 5C). Similarly, CCW12(−) SECReTE cells exhibited hypersensitivity to HB (FIG. 5E).

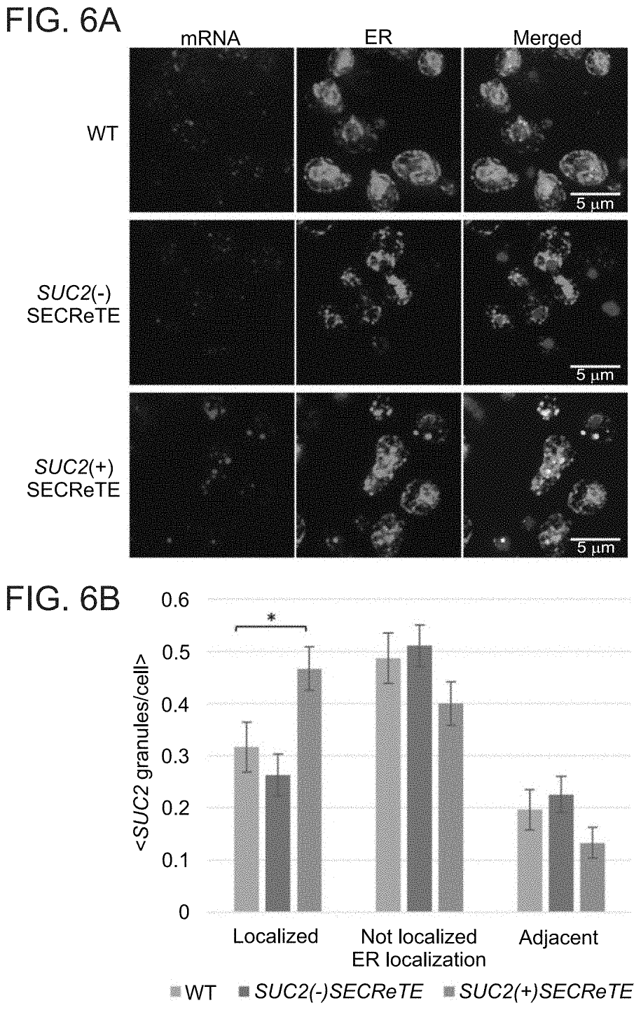

These findings strengthen the notion that SECReTE plays an important biological role in regulating the amount of protein secreted from cells. This was verified using an exogenous substrate, SS(GAS1)-GFP, whose secretion was significantly enhanced upon addition of the Gas 1 3′UTR containing the SECReTE motif (FIG. 5F). Moreover, strengthening of SECReTE not only increased protein production and secretion, it also enhanced the localization of SUC2 transcripts to the ER (FIG. 6A-B).

Consequently, the present teachings suggest that this motif may be used to improve recombinant protein production.

Thus, according to a first aspect of the present invention, there is provided an isolated polynucleotide comprising a transcriptional unit, wherein the transcriptional unit comprises:

(i) a nucleic acid sequence that encodes a secreted protein of interest;

(ii) an endoplasmic reticulum (ER) targeting sequence as set forth in SEQ ID NO: 2, said ER targeting sequence being heterologous to said secreted protein of interest;

(iii) a promoter; and

(iv) a transcription termination site, wherein said nucleic acid sequence that encodes a protein of interest and said ER targeting sequence are positioned between said promoter and said transcription termination site;

wherein when said ER targeting sequence is comprised in said nucleic acid sequence that encodes said protein of interest, said nucleic acid sequence has been codon optimized to comprise said ER targeting sequence.

The phrase “an isolated polynucleotide” refers to a single or double stranded nucleic acid sequence which is provided in the form of an isolated DNA molecule (i.e. comprising deoxyribonucleotides).

The term “transcriptional unit” refers to a sequence of DNA that codes for a single RNA molecule, together with the sequences necessary for its transcription. Typically, the transcriptional unit contains a promoter, a sequence that encodes for a protein of interest and a terminator.

Each of these elements will be described individually herein below.

Nucleic acid sequence that encodes a protein of interest:

The proteins of interest are typically secreted proteins. In one embodiment, the proteins are human proteins, although the present invention contemplates proteins from other species as well.

Exemplary proteins of interest that can be produced by employing the subject compositions and methods include but are not limited to certain native and recombinant human hormones (e.g., insulin, growth hormone, insulin-like growth factor 1, follicle-stimulating hormone, and chorionic gonadotropin), hematopoietic proteins (e.g., erythropoietin, C-CSF, GM-CSF, and IL-11), thrombotic and hematostatic proteins (e.g., tissue plasminogen activator and activated protein C), immunological proteins (e.g., interleukin), antibodies and other enzymes (e.g., deoxyribonuclease I). Exemplary vaccines that can be produced by the subject compositions and methods include but are not limited to vaccines against various influenza viruses (e.g., types A, B and C and the various serotypes for each type such as H5N2, H1N1, H3N2 for type A influenza viruses), HIV, hepatitis viruses (e.g., hepatitis A, B, C or D), Lyme disease, and human papillomavirus (HPV). Examples of heterologously produced protein diagnostics include but are not limited to secretin, thyroid stimulating hormone (TSH), HIV antigens, and hepatitis C antigens.

Other exemplary proteins of interest can include, but are not limited to cytokines, chemokines, lymphokines, ligands, receptors, hormones, enzymes, antibodies and antibody fragments, and growth factors. Non-limiting examples of receptors include TNF type I receptor, IL-1 receptor type II, IL-1 receptor antagonist, IL-4 receptor and any chemically or genetically modified soluble receptors. Examples of enzymes include acetlycholinesterase, lactase, activated protein C, factor VII, collagenase (e.g., marketed by Advance Biofactures Corporation under the name Santyl); agalsidase-beta (e.g., marketed by Genzyme under the name Fabrazyme); dornase-alpha (e.g., marketed by Genentech under the name Pulmozyme); alteplase (e.g., marketed by Genentech under the name Activase); pegylated-asparaginase (e.g., marketed by Enzon under the name Oncaspar); asparaginase (e.g., marketed by Merck under the name Elspar); and imiglucerase (e.g., marketed by Genzyme under the name Ceredase). Examples of specific polypeptides or proteins include, but are not limited to collagen, granulocyte macrophage colony stimulating factor (GM-CSF), granulocyte colony stimulating factor (G-CSF), macrophage colony stimulating factor (M-CSF), colony stimulating factor (CSF), interferon beta (IFN-beta), interferon gamma (IFNgamma), interferon gamma inducing factor I (IGIF), transforming growth factor beta (IGF-beta), RANTES (regulated upon activation, normal T-cell expressed and presumably secreted), macrophage inflammatory proteins (e.g., MIP-1-alpha and MIP-1-beta), Leishmnania elongation initiating factor (LEIF), platelet derived growth factor (PDGF), tumor necrosis factor (TNF), growth factors, e.g., epidermal growth factor (EGF), vascular endothelial growth factor (VEGF), fibroblast growth factor, (FGF), nerve growth factor (NGF), brain derived neurotrophic factor (BDNF), neurotrophin-2 (NT-2), neurotrophin-3 (NT-3), neurotrophin-4 (NT-4), neurotrophin-5 (NT-5), glial cell line-derived neurotrophic factor (GDNF), ciliary neurotrophic factor (CNTF), TNF alpha type II receptor, erythropoietin (EPO), insulin and soluble glycoproteins e.g., gp120 and gp160 glycoproteins. The gp120 glycoprotein is a human immunodeficiency virus (WIV) envelope protein, and the gp160 glycoprotein is a known precursor to the gp120 glycoprotein. Other examples include secretin, nesiritide (human B-type natriuretic peptide (hBNP)) and GYP-I.

Other exemplary proteins of interest may include GPCRs, including, but not limited to Class A Rhodopsin like receptors such as Muscatinic (Muse.) acetylcholine Vertebrate type 1, Musc. acetylcholine Vertebrate type 2, Musc. acetylcholine Vertebrate type 3, Musc. acetylcholine Vertebrate type 4; Adrenoceptors (Alpha Adrenoceptors type 1, Alpha Adrenoceptors type 2, Beta Adrenoceptors type 1, Beta Adrenoceptors type 2, Beta Adrenoceptors type 3, Dopamine Vertebrate type 1, Dopamine Vertebrate type 2, Dopamine Vertebrate type 3, Dopamine Vertebrate type 4, Histamine type 1, Histamine type 2, Histamine type 3, Histamine type 4, Serotonin type 1, Serotonin type 2, Serotonin type 3, Serotonin type 4, Serotonin type 5, Serotonin type 6, Serotonin type 7, Serotonin type 8, other Serotonin types, Trace amine, Angiotensin type 1, Angiotensin type 2, Bombesin, Bradykffin, C5a anaphylatoxin, Finet-leu-phe, APJ like, Interleukin-8 type A, Interleukin-8 type B, Interleukin-8 type others, C-C Chemokine type 1 through type 11 and other types, C-X-C Chemokine (types 2 through 6 and others), C-X3-C Chemokine, Cholecystokinin CCK, CCK type A, CCK type B, CCK others, Endothelin, Melanocortin (Melanocyte stimulating hormone, Adrenocorticotropic hormone, Melanocortin hormone), Duffy antigen, Prolactin-releasing peptide (GPR10), Neuropeptide Y (type 1 through 7), Neuropeptide Y, Neuropeptide Y other, Neurotensin, Opioid (type D, K, M, X), Somatostatin (type 1 through 5), Tachykinin (Substance P(NK1), Substance K (NK2), Neuromedin K (NK3), Tachykinin like 1, Tachykinin like 2, Vasopressin/vasotocin (type 1 through 2), Vasotocin, Oxytocin/mesotocin, Conopres sin, Galanin like, Proteinase-activated like, Orexin & neuropeptides FF, QRFP, Chemokine receptor-like, Neuromedin U like (Neuromedin U, PRXamide), hormone protein (Follicle stimulating hormone, Lutropin-choriogonadotropic hormone, Thyrotropin, Gonadotropin type I, Gonadotropin type II), (Rhod)opsin, Rhodopsin Vertebrate (types 1-5), Rhodopsin Vertebrate type 5, Rhodopsin Arthropod, Rhodopsin Arthropod type 1, Rhodopsin Arthropod type 2, Rhodopsin Arthropod type 3, Rhodopsin Mollusc, Rhodopsin, Olfactory (Olfactory 11 fam 1 through 13), Prostaglandin (prostaglandin E2 subtype EP 1, Prostaglandin E2/D2 subtype EP2, prostaglandin E2 subtype EP3, Prostaglandin E2 subtype EP4, Prostaglandin F2-alpha, Prostacyclin, Thromboxane, Adenosine type 1 through 3, Purinoceptors, Purinoceptor P2RY1-4,6,11 GPR91, Purinoceptor P2RY5,8,9,10 GPR35,92,174, Purinoceptor P2RY12-14 GPR87 (JDP-Glucose), Cannabinoid, Platelet activating factor, Gonadotropin-releasing hormone, Gonadotropin-releasing hormone type I, Gonadotropin-releasing hormone type II, Adipokinetic hormone like, Corazonin, Thyrotropin-releasing hormone & Secretagogue, Thyrotropin-releasing hormone, Growth hormone secretagogue, Growth hormone secretagogue like, Ecdysis-triggering hormone (ETHR), Melatonin, Lysosphingolipid & LPA (EDG), Sphingosine 1-phosphate Edg-1, Lysophosphatidic acid Edg-2, Sphingosine 1-phosphate Edg-3, Lysophosphatidic acid Edg4, Sphingosine 1-phosphate Edg-5, Sphingosine 1-phosphate Edg-6, Lysophosphatidic acid Edg-7, Sphingosine 1-phosphate Edg-8, Edg Other Leukotriene B4 receptor, Leukotriene B4 receptor BLT1, Leukotriene B4 receptor BLT2, Class A Orphan/other, Putative neurotransmitters, SREB, Mas proto-oncogene & Mas-related (MRGs), GPR45 like, Cysteinyl leukotriene, G-protein coupled bile acid receptor, Free fatty acid receptor (GP40, GP41, GP43), Class B Secretin like, Calcitonin, Corticotropin releasing factor, Gastric inhibitory peptide, Glucagon, Growth hormone-releasing hormone, Parathyroid hormone, PACAP, Secretin, Vasoactive intestinal polypeptide, Latrophilin, Latrophilin type 1, Latrophilin type 2, Latrophilin type 3, ETL receptors, Brain-specific angiogenesis inhibitor (BAI), Methuselah-like proteins (MTH), Cadherin EGF LAG (CELSR), Very large G-protein coupled receptor, Class C Metabotropic glutamate/pheromone, Metabotropic glutamate group I through III, Calcium-sensing like, Extracellular calcium-sensing, Pheromone, calcium-sensing like other, Putative pheromone receptors, GABA-B, GABA-B subtype 1, GABA-B subtype 2, GABA-B like, Orphan GPRC5, Orphan GPCR6, Bride of sevenless proteins (BOSS), Taste receptors (TiR), Class D Fungal pheromone, Fungal pheromone A-Factor like (STE2,STE3), Fungal pheromone B like (BAR,BBR,RCB,PRA), Class E cAMP receptors, Ocular albinism proteins, Frizzled/Smoothened family, frizzled Group A (Fz 1&2&4&5&7-9), frizzled Group B (Fz 3 & 6), fizzled Group C (other), Vomeronasal receptors, Nematode chemoreceptors, Insect odorant receptors, and Class Z Archaeal/bacterial/fungal opsins.

The polypeptide of interest may also be a bioactive peptide. Examples include: BOTOX, Myobloc, Neurobloc, Dysport (or other serotypes of botulinum neurotoxins), alglucosidase alfa, daptomycin, YH-16, choriogonadotropin alfa, filgrastim, cetrorelix, interleukin-2, aldesleukin, teceleulin, denileukin diftitox, interferon alfa-n3 (injection), interferon alfa-n1, DL-8234, interferon, Suntory (gamma-1a), interferon gamma, thymosin alpha 1, tasonermin, DigiFab, ViperaTAb, EchiTAb, CroFab, nesiritide, abatacept, alefacept, Rebif, eptoterminalfa, teriparatide (osteoporosis), calcitonin injectable (bone disease), calcitonin (nasal, osteoporosis), etanercept, hemoglobin glutamer 250 (bovine), drotrecogin alfa, collagenase, carperitide, recombinant human epidermal growth factor (topical gel, wound healing), DWP401, darbepoetin alfa, epoetin omega, epoetin beta, epoetin alfa, desirudin, lepirudin, bivalirudin, nonacog alpha, Mononine, eptacog alfa (activated), recombinant Factor VIII+VWF, Recombinate, recombinant Factor VIII, Factor VIII (recombinant), Alphnmate, octocog alfa, Factor VIII, palifermin, Indikinase, tenecteplase, alteplase, pamiteplase, reteplase, nateplase, monteplase, follitropin alfa, rFSH, hpFSH, micafungin, pegfilgrastim, lenograstim, nartograstim, sermorelin, glucagon, exenatide, pramlintide, iniglucerase, galsulfase, Leucotropin, molgramostim, triptorelin acetate, histrelin (subcutaneous implant, Hydron), deslorelin, histrelin, nafarelin, leuprolide sustained release depot (ATRIGEL), leuprolide implant (DUROS), goserelin, somatropin, Eutropin, KP-102 program, somatropin, somatropin, mecasermin (growth failure), enlfavirtide, Org-33408, insulin glargine, insulin glulisine, insulin (inhaled), insulin lispro, insulin deternir, insulin (buccal, RapidMist), mecasermin rinfabate, anakinra, celmoleukin, 99 mTc-apcitide injection, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex' recombinant human luteinizing hormone, dornase alfa, trafermin, ziconotide, taltirelin, diboterminalfa, atosiban, becaplermin, eptifibatide, Zemaira, CTC-111, Shanvac-B, HPV vaccine (quadrivalent), octreotide, lanreotide, ancestirn, agalsidase beta, agalsidase alfa, laronidase, prezatide copper acetate (topical gel), rasburicase, ranibizumab, Actimmune, PEG-Intron, Tricomin, recombinant house dust mite allergy desensitization injection, recombinant human parathyroid hormone (PTH) 1-84 (sc, osteoporosis), epoetin delta, transgenic antithrombin III, Granditropin, Vitrase, recombinant insulin, interferon-alpha (oral lozenge), GEM-21S, vapreotide, idursulfase, omnapatrilat, recombinant serum albumin, certolizumab pegol, glucarpidase, human recombinant C1 esterase inhibitor (angioedema), lanoteplase, recombinant human growth hormone, enfuvirtide (needle-free injection, Biojector 2000), VGV-1, interferon (alpha), lucinactant, aviptadil (inhaled, pulmonary disease), icatibant, ecallantide, omiganan, Aurograb, pexigananacetate, ADI-PEG-20, LDI-200, degarelix, cintredelinbesudotox, Favld, MDX-1379, ISAtx-247, liraglutide, teriparatide (osteoporosis), tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone (sustained release injection), recombinant G-CSF, insulin (inhaled, AIR), insulin (inhaled, Technosphere), insulin (inhaled, AERx), RGN-303, DiaPep277, interferon beta (hepatitis C viral infection (HCV)), interferon alfa-n3 (oral), belatacept, transdermal insulin patches, AMG-531, MBP-8298, Xerecept, opebacan, AIDSVAX, GV-1001, LymphoScan, ranpirnase, Lipoxysan, lusupultide, MP52 (beta-tricalciumphosphate carrier, bone regeneration), melanoma vaccine, sipuleucel-T, CTP-37, Insegia, vitespen, human thrombin (frozen, surgical bleeding), thrombin, TransMID, alfimeprase, Puricase, terlipressin (intravenous, hepatorenal syndrome), EUR-1008M, recombinant FGF-I (injectable, vascular disease), BDM-E, rotigaptide, ETC-216, P-113, MBI-594AN, duramycin (inhaled, cystic fibrosis), SCV-07, OPI-45, Endostatin, Angiostatin, ABT-510, Bowman Birk Inhibitor Concentrate, XMP-629, 99 mTc-Hynic-Annexin V, kahalalide F, CTCE-9908, teverelix (extended release), ozarelix, rornidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, ovarian cancer immunotherapeutic vaccine, SB-249553, Oncovax-CL, OncoVax-P, BLP-25, CerVax-16, multi-epitope peptide melanoma vaccine (MART-1, gp100, tyrosinase), nemifitide, rAAT (inhaled), rAAT (dermatological), CGRP (inhaled, asthma), pegsunercept, thymosinbeta4, plitidepsin, GTP-200, ramoplanin, GRASPA, OBI-1, AC-100, salmon calcitonin (oral, eligen), calcitonin (oral, osteoporosis), examorelin, capromorelin, Cardeva, velafermin, 131I-TM-601, KK-220, T-10, ularitide, depelestat, hematide, Chrysalin (topical), rNAPc2, recombinant Factor V111 (PEGylated liposomal), bFGF, PEGylated recombinant staphylokinase variant, V-10153, SonoLysis Prolyse, NeuroVax, CZEN-002, islet cell neogenesis therapy, rGLP-1, BIM-51077, LY-548806, exenatide (controlled release, Medisorb), AVE-0010, GA-GCB, avorelin, AOD-9604, linaclotid eacetate, CETi-1, Hemospan, VAL (injectable), fast-acting insulin (injectable, Viadel), intranasal insulin, insulin (inhaled), insulin (oral, eligen), recombinant methionyl human leptin, pitrakinra subcutancous injection, eczema), pitrakinra (inhaled dry powder, asthma), Multikine, RG-1068, MM-093, NBI-6024, AT-001, PI-0824, Org-39141, Cpn10 (autoimmune diseases/inflammation), talactoferrin (topical), rEV-131 (ophthalmic), rEV-131 (respiratory disease), oral recombinant human insulin (diabetes), RPI-78M, oprelvekin (oral), CYT-99007 CTLA4-Ig, DTY-001, valategrast, interferon alfa-n3 (topical), IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, malaria vaccine (virosomes, PeviPRO), ALTU-135, parvovirus B19 vaccine, influenza vaccine (recombinant neuraminidase), malaria/HBV vaccine, anthrax vaccine, Vacc-5q, Vacc-4x, HIV vaccine (oral), HPV vaccine, Tat Toxoid, YSPSL, CHS-13340, PTH(1-34) liposomal cream (Novasome), Ostabolin-C, PTH analog (topical, psoriasis), MBRI-93.02, MTB72F vaccine (tuberculosis), MVA-Ag85A vaccine (tuberculosis), FARA04, BA-210, recombinant plague F1V vaccine, AG-702, OxSODrol, rBetV1, Der-p1/Der-p2/Der-p7 allergen-targeting vaccine (dust mite allergy), PR1 peptide antigen (leukemia), mutant ras vaccine, HPV-16 E7 lipopeptide vaccine, labyrinthin vaccine (adenocarcinoma), CML vaccine, WT1-peptide vaccine (cancer), IDD-5, CDX-110, Pentrys, Norelin, CytoFab, P-9808, VT-111, icrocaptide, telbermin (dermatological, diabetic foot ulcer), rupintrivir, reticulose, rGRF, HA, alpha-galactosidase A, ACE-011, ALTU-140, CGX-1160, angiotensin therapeutic vaccine, D-4F, ETC-642, APP-018, rhMBL, SCV-07 (oral, tuberculosis), DRF-7295, ABT-828, ErbB2-specific immunotoxin (anticancer), DT3SSIL-3, TST-10088, PRO-1762, Combotox, cholecystokinin-B/gastrin-receptor binding peptides, 111In-hEGF, AE-37, trasnizumab-DM1, Antagonist G, IL-12 (recombinant), PM-02734, IMP-321, rhIGF-BP3, BLX-883, CUV-1647 (topical), L-19 based radioimmunotherapeutics (cancer), Re-188-P-2045, AMG-386, DC/1540/KLH vaccine (cancer), VX-001, AVE-9633, AC-9301, NY-ESO-1 vaccine (peptides), NA17.A2 peptides, melanoma vaccine (pulsed antigen therapeutic), prostate cancer vaccine, CBP-501, recombinant human lactoferrin (dry eye), FX-06, AP-214, WAP-8294A (injectable), ACP-HIP, SUN-11031, peptide YY [3-36] (obesity, intranasal), FGLL, atacicept, BR3-Fc, BN-003, BA-058, human parathyroid hormone 1-34 (nasal, osteoporosis), F-18-CCR1, AT-1100 (celiac disease/diabetes), JPD-003, PTH (7-34) liposomal cream (Novasome), duramycin (ophthalmic, dry eye), CAB-2, CTCE-0214, GlycoPEGylated erythropoietin, EPO-Fc, CNTO-528, AMG-114, JR-013, Factor XIII, aminocandin, PN-951, 716155, SUN-E7001, TH-0318, BAY-73-7977, teverelix (immediate release), EP-51216, hGH (controlled release, Biosphere), OGP-I, sifuvirtide, TV4710, ALG-889, Org-41259, rhCC10, F-991, thymopentin (pulmonary diseases), r(m)CRP, hepatoselective insulin, subalin, L19-IL-2 fusion protein, elafin, NMK-150, ALTU-139, EN-122004, rhTPO, thrombopoietin receptor agonist (thrombocytopenic disorders), AL-108, AL-208, nerve growth factor antagonists (pain), SLV-317, CGX-1007, INNO-105, oral teriparatide (eligen), GEM-OS1, AC-162352, PRX-302, LFn-p24 fusion vaccine (Therapore), EP-1043, S pneumoniae pediatric vaccine, malaria vaccine, Neisseria meningitidis Group B vaccine, neonatal group B streptococcal vaccine, anthrax vaccine, HCV vaccine (gpE1+gpE2+MF-59), otitis media therapy, HCV vaccine (core antigen+ISCOMATRIX), hPTH (1-34) (transdermal, ViaDerm), 768974, SYN-101, PGN-0052, aviscumnine, BIM-23190, tuberculosis vaccine, multi-epitope tyrosinase peptide, cancer vaccine, enkastim, APC-8024, GI-5005, ACC-001, TTS-CD3, vascular-targeted TNF (solid tumors), desmopressin (buccal controlled-release), onercept, and TP-9201.

In certain embodiments, the protein of interest is an enzyme or biologically active fragments thereof. Suitable enzymes include but are not limited to: oxidoreductases, transferases, hydrolases, lyases, isomerases, and ligases. In certain embodiments, the heterologously produced protein is an enzyme of Enzyme Commission (EC) class 1, for example an enzyme from any of EC 1.1 through 1.21, or 1.97. The enzyme can also be an enzyme from EC class 2, 3, 4, 5, or 6. For example, the enzyme can be selected from any of EC 2.1 through 2.9, EC 3.1 to 3.13, EC 4.1 to 4.6, EC 4.99, EC 5.1 to 5.11, EC 5.99, or EC 6.1-6.6.

In another embodiment, the protein of interest is an antibody.

As used herein, the term “antibody” refers to a substantially intact antibody molecule.

As used herein, the phrase “antibody fragment” refers to a functional fragment of an antibody (such as Fab, F(ab′)2, Fv or single domain molecules such as VH and VL) that is capable of binding to an epitope of an antigen.

Promoter:

As used herein, the term “promoter” refers to any nucleic acid sequence, such as a DNA sequence, which is recognized and bound (directly or indirectly) by a DNA-dependent RNA-polymerase during initiation of transcription, resulting in the generation of an RNA molecule that is complementary to the transcribed DNA. Promoters are usually located upstream of the 5′ untranslated region (UTR) preceding the protein coding sequence to be transcribed and have regions that act as binding sites for RNA polymerase II and other proteins such as transcription factors to initiate transcription of an operably linked sequence. Promoters may themselves contain sub-elements (i.e. promoter motifs) such as cis-elements or enhancer domains that regulate the transcription of operably linked genes. The promoter and a connected 5′ UTR are also referred to as “promoter region”.

The promoter of this aspect of the present invention may be constitutive or inducible.

Constitutive promoters suitable for use with this embodiment of the present invention include sequences which are functional (i.e., capable of directing transcription) under most environmental conditions and most types of cells such as the cytomegalovirus (CMV), SV-40 early promoter, SV-40 later promoter, metallothionein promoter, murine mammary tumor virus promoter, Rous sarcoma virus promoter and polyhedrin promoter.

Inducible promoters suitable for use with this embodiment of the present invention include for example the tetracycline-inducible promoter (Srour, M. A., et al., 2003. Thromb. Haemost. 90: 398-405) or IPTG.

In yeast, a number of constitutive or inducible promoters can be used, as disclosed in U.S. Pat. No. 5,932,447. Alternatively, vectors can be used which promote integration of foreign DNA sequences into the yeast chromosome.

In cases where expression in plants is required, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used.

Transcription termination site:

As used herein, the term “transcription termination site” refers to a DNA sequence directing the transcription termination by RNA polymerase. Said sequences can also direct post-transcriptional cleavage and polyadenylation of transcribed RNA. In a particular embodiment, the transcription termination sequence comprises a polyadenylation signal, referred to as polyadenylation/termination sequence. In a preferred embodiment, the termination sequence is derived from SV40 virus.

Endoplasmic reticulum (ER) targeting sequence as set forth in SEQ ID NO: 2:

The ER targeting sequence of this aspect of the present invention aids in localizing the attached sequence to the ER. In one embodiment, the ER targeting sequence promotes uptake of the attached sequence into the ER.

The NNY repeat of this sequence is repeated at least 7 times (as in SEQ ID NO: 1), eight times, nine times, ten times, 11 times, 12 times. 13 times, 14 times, 15 times, 16 times, 17 times, 18 times, 19 times, 20 times or more.

According to a particular embodiment, the NNY repeat is repeated at least 10 times (as in SEQ ID NO: 2).

In a particular embodiment, the N of the NNY repeat is a pyrimidine. In another embodiment, the N of the NNY repeat is a purine. In all embodiments, the Y of the NNY repeat is a pyrimidine.

According to a particular embodiment, the ER targeting sequence does not comprise nucleotides that encode for a protein of interest.

According to still another embodiment, the ER targeting sequence does not comprise nucleotides that encode for a sequence as set forth in SEQ ID NO: 5. (KDEL).

Preferably, the ER targeting sequence does not comprise the sequence as set forth in SEQ ID NO: 6 (agc tacacccacc acctcatcta cctctac),

Furthermore, in one embodiment, the ER targeting sequence does not comprise more than 5 consecutive repeats of the sequence TG.

Preferably, the ER targeting sequence does not comprise more than 10, 15, 20, 25, 30 or more consecutive thymines.

Preferably, the ER targeting sequence does not comprise more than 10, 15, 20, 25, 30 or more consecutive cytosines.

The ER targeting sequence is heterologous (i.e. not endogenous) to the secreted protein of interest—i.e. it is not part of the native sequence which encodes (or regulates expression of) the protein of interest.

The positioning of each of these elements is such that the nucleic acid sequence that encodes the protein of interest and the ER targeting sequence are positioned between the promoter and the transcription termination site. In this way an mRNA transcript is generated which encodes the protein of interest and further comprises the ER targeting sequence.

In one embodiment, the ER targeting sequence of this aspect of the present invention is positioned 3′ to the sequence that encodes the protein of interest. In another embodiment the ER targeting sequence of this aspect of the present invention is positioned 5′ to the sequence that encodes the protein of interest. In still further embodiments, the ER targeting sequence is encoded in the sequence that encodes for the protein of interest.

Preferably, when the ER targeting sequence is comprised in the nucleic acid sequence that encodes the protein of interest, the nucleic acid sequence of the protein is codon optimized to comprise the ER targeting sequence. Thus, the amino acid sequence of the protein of interest is identical to the native amino acid sequence.

The phrase “codon optimization” refers to the selection of appropriate DNA nucleotides for use within a structural gene or fragment thereof such that the ER targeting sequence is encoded within the DNA sequence without affecting the amino acid sequence of the protein. Therefore, an optimized gene or nucleic acid sequence refers to a gene in which the nucleotide sequence of a native or naturally occurring gene has been modified in order to utilize codons which comprise the ER targeting sequence.

The transcriptional unit of this aspect of the present invention may further comprise a sequence that encodes a signal peptide sequence.

As used herein, the phrase “signal peptide” refers to a peptide linked in frame to the amino terminus of a polypeptide and directs the encoded polypeptide into a cell's secretory pathway.

The signal sequence is typically located N-terminal to the protein sequence. A signal sequence is normally absent from the mature protein. A signal sequence is typically cleaved from the protein by a signal peptidase after the protein is transported.

According to one embodiment, the polypeptide encodes a signal peptide having a sequence as set forth in SEQ ID NO: 3 (ATGTTGTTTAAATCCCTTTCAAAGTTAGCAACCGCTGCTGCTTTTTTTGCTGGCGTCG CAACTGCGGAC).

In one embodiment, the signal peptide sequence is endogenous to the protein of interest. In another embodiment, the signal peptide sequence is heterologous (i.e. is not native, or is exogenous, non-endogenous) to the protein of interest.

A variety of prokaryotic or eukaryotic cells can be used as host-expression systems to express the polypeptides of the present invention. These include, but are not limited to, bacterial cells (e.g. E. coli), fungal cells (e.g. S. cerevisiae cells), plant cells (e.g. tobacco), insect cells (lepidopteran cells) and other mammalian cells (Chinese Hamster Ovary cells).

The cells may be part of a cell culture, a whole organism, or a part of an organism.

The term “plant” as used herein encompasses whole plants, ancestors and progeny of the plants and plant parts, including seeds, shoots, stems, roots (including tubers), and plant cells, tissues and organs. The plant may be in any form including suspension cultures, embryos, meristematic regions, callus tissue, leaves, gametophytes, sporophytes, pollen, and microspores. Plants that are particularly useful in the methods of the invention include all plants which belong to the superfamily Viridiplantae, in particular monocotyledonous and dicotyledonous plants including a fodder or forage legume, ornamental plant, food crop, tree, or shrub. Algae and other non-Viridiplantae can also be used for the methods of the present invention.

Contemplated cells for the expression of human interferon beta 1a include for example Chinese Hamster Ovary (CHO) cells.

Contemplated cells for the expression of human interferon beta 1b include for example E.coli cells.

Contemplated cells for the expression of human interferon gamma include for example E. coli cells.

Contemplated cells for the expression of human growth hormone include for example E. coli cells.

Contemplated cells for the expression of human insulin include for example E. coli cells.

Contemplated cells for the expression of interleukin II include for example E. coli cells.

Contemplated cells for the expression of follicle stimulating hormone include for example CHO cells.

In order to express the polypeptides from the polynucleotides of the present invention in cell systems, the polynucleotides are ligated into nucleic acid expression vectors.

The expression vector according to this embodiment of the present invention may include additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals).

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

Polyadenylation sequences can also be added to the expression vector in order to increase the translation efficiency of a polypeptide expressed from the expression vector of the present invention. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Expression vectors containing regulatory elements from eukaryotic viruses such as retroviruses can also be used by the present invention. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, N.Y., Section VIII, pp 421-463.

Examples of mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In one embodiment, the expression vector comprises a nucleic acid sequence which comprises the ER targeting sequence of the present invention (e.g. as set forth in SEQ ID NO: 1 or SEQ ID NO: 2) and a cloning site, wherein a position of the cloning site is selected such that upon insertion of a sequence which encodes a protein of interest into the cloning site, following expression in a cell, an mRNA is transcribed which encodes the protein of interest, wherein the ER targeting sequence is not part of a sequence that encodes for a known protein of interest—e.g. does not encode for MRL1-3; as disclosed in Kraut Cohen et al Mol. Biol. Cell 24, 3069-3084. The synthesized mRNA comprises a transcription product of the ER targeting sequence.

The term “cloning site” refers to a location on a vector into which DNA can be inserted. The term “multiple cloning site” or “mcs” refers to a synthetic DNA sequence that contains any one or a number of different restriction enzyme sites to permit insertion at a defined locus (the restriction site) on a vector. The term “unique cloning site” refers to a cloning site that appears one time with a given DNA sequence.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Transformed cells are cultured under effective conditions, which allow for the expression of high amounts of recombinant polypeptide. Effective culture conditions include, but are not limited to, effective media, bioreactor, temperature, pH and oxygen conditions that permit protein production. An effective medium refers to any medium in which a cell is cultured to produce the recombinant polypeptide of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes and petri plates. Culturing can be carried out at a temperature, pH and oxygen content appropriate for a recombinant cell. Such culturing conditions are within the expertise of one of ordinary skill in the art.

Following a predetermined time in culture, recovery of the recombinant polypeptide is affected.

The phrase “recovering the recombinant polypeptide” used herein refers to collecting the whole fermentation medium containing the polypeptide and need not imply additional steps of separation or purification.

Thus, polypeptides of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.
Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

### Yeast Strains, Genomic Manipulations, and Growth Conditions

Yeasts were grown at the indicated temperature either in a standard growth medium (1% Yeast Extract, 2% Peptone, 2% Dextrose) or synthetic medium containing 2% glucose [e.g., synthetic complete (SC) and selective SC dropout medium lacking an amino acid or nucleotide base] (Haim and Gerst, 2009). Deletion strains using the NAT antibiotic resistance gene in WT (BY4741) cells were created using standard LiOAc transformation procedures and with nourseothricin (100 μg/ml) for selection on synthetic solid medium. For the creation of SECReTE mutant strains, SECReTE gene fragments were designed with the appropriate modifications, from the first to the last mutated base, and synthesized either as a gBlock™ (Integrated DNA Technologies, Inc., Coralville, Iowa, USA) or cloned into a pUC57-AMP vector (Bio Basic Inc.). Both (−)SECReTE and (+)SECReTE strains were generated. SUC2(−)SECReTE, SUC2(+)SECReTE and CCW/2(−)SECReTE strains were constructed in the BY4741 background genome using the delitto perfetto method for genomic oligonucleotide recombination (Storici and Resnick, 2006), in which the CORE cassette from pGKSU (Storici and Resnick, 2006) was integrated first into the genomic region corresponding to site of the SECReTE gene fragment. The CORE cassette contains the URA3 selection marker with an I-SceI homing endonuclease site and a separate inducible I-SceI gene. The SECReTE gene fragment for CCW/2(−)SECReTE was amplified from the synthetic gBlock using primer sequences containing 20 bases of homology to both the region outside of the desired genomic locus and the CORE cassette. The amplified SECReTE gene fragment subsequently replaced the CORE cassette in the desired genomic site through an additional step of integration. CRISPR/Cas9 was utilized instead to generate the HSP150 mutant strains. HSP150(−)SECReTE and HSP150(+)SECReTE were created in the BY4741 genome. The CRISPR/Cas9 procedure involved deletion of the native genomic region corresponding to the SECReTE gene fragment, using the NAT cassette from pFA6-NatMX6. A CRISPR/Cas9 plasmid vector was designed to express the Cas9 gene, a guide RNA that targets the NAT cassette, and the LEU2 selection marker. The CRISPR/Cas9 plasmid was co-transformed with the amplified SECReTE gene fragment to replace the NAT cassette. Standard LiOAc-based protocols were employed for transformations of plasmids and PCR products into yeast. Transformed cells were then grown for 2-4 days on selective media. Correct integrations were verified at each step using PCR and, at the final step, accurate integration of the (−)SECReTE or (+)SECReTE sequences was confirmed by DNA sequencing.

### Quantitative RT-PCR (qRT-PCR)

RNA was extracted and purified from overnight cultures using a MasterPure Yeast RNA Purification kit (Epicentre Biotechnologies). For each sample, 2 μg of purified RNA was treated with DNase (Promega, Madison, Wis., USA) for 2 hrs at 37° C. and subjected to reverse transcription (RT) using Moloney murine leukemia virus RT RNase H(−) (Promega) under the recommended manufacturer conditions. Primer pairs were designed, using NCBI Primer-Blast (Ye et al., 2012), to produce only one amplicon (60-70 bp). Standard curves were generated for each pair of primers and primer efficiency was measured. All sets of reactions were conducted in triplicate and each included a negative control (H₂O). qRT-PCR was performed using a LightCycler® 480 device and SYBR® Green PCR Master Mix (Applied Biosystems®, Waltham, Mass., USA). Two-step qRT-PCR thermocycling parameters were used as specified by the manufacturer. Analysis of the melting curve assessed the specificity of individual real-time PCR products and revealed a single peak for each real-time PCR product. The ACT1 or UBC6 RNAs were used for normalization and fold-change was calculated relative to WT cells.

### Drop Test Growth Assays

Drop test assays were performed by growing yeast strains in YPD medium to mid-log phase and then performing serial dilution five times (10-fold each) in fresh medium. Cells were spotted onto plates with different conditions and incubated for 48 hrs, prior to photo-documentation. Calcofluor White (CFW) or Hygromycin B (HB) sensitivity was tested by spotting cells onto YPD plates containing either 25 μg/ml HB or 50 μg/ml CFW (dissolved in DMSO, prepared as in Ram and Klis (2006)), following the protocol as mentioned above.

### Hsp150 and GFP Secretion Assays

For the induction of Hsp150 secretion, strains were grown in YPD overnight at 26° C., diluted in YPD medium to 0.2 0.D.600 units, and then incubated at 37° C. and grown until log-phase. For GFP secretion, yeast were grown O/N to 0.2 0.D.₆₀₀ at 30° C. in synthetic selective medium containing raffinose as a carbon source, then diluted to 0.2 OD O.D.₆₀₀ units in YP-Gal, and grown to mid-log phase (0.6-0.8 O.D.₆₀₀) at 30° C. Next, 1.8 ml of the culture was taken from each strain and centrifuged for 3 minutes at 1900×g at room temperature. Trichloroacetic Acid (100% w/v) protein precipitation was performed on the supernatant and protein extraction, using NaOH 0.1M, was performed on the pellet (Zhang et al., 2011). Samples were separated on SDS-PAGE gels, blotted electrophoretically onto nitrocellulose membranes, and detected by incubation with rabbit anti-Hsp150 [1:10,000 dilution; gift from Jussi Jäntti (VTT Research, Helsinki)] or monoclonal mouse anti-GFP (Roche Applied Science, Penzberg, Germany) antibodies followed by visualisation using the Enhanced Chemiluminescence (ECL) detection system with anti-rabbit peroxidase-conjugated antibodies (1:10,000, Amersham Biosciences). Protein markers (ExcelBand 3-color Broad Range Protein Marker PM2700, SMOBiO Technology, Inc., Hsinchu, Taiwan) were used to assess protein molecular mass.

### Invertase Assay

Invertase secretion was measured as described previously (Goldstein and Lampen, 1975). Cell preparation for the invertase assay was performed as described in (Novick and Schekman, 1979). The protocol was optimized based on (Troy A A, 2014). Internal and external activities were expressed in units based on absorption at 540 nm (1 U=1 μmol glucose released/min per OD unit).

### Single-Molecule FISH

Yeast cells expressing Sec63-GFP were grown to mid log phase and shifted to low glucose-containing medium [0.1% glucose] for 1.5 h to induce SUC2 expression. Cells were fixed in the same medium upon the addition of formaldehyde (3.7% final concentration) for 45 min. Cells were gently washed twice with 0.1M potassium phosphate buffer, pH 7.4 containing 1.2M sorbitol, after which cells were spheroplasted in 1 ml of freshly prepared spheroplast buffer [0.1M potassium phosphate buffer, pH 7.4, 1.2M sorbitol, 20 mM ribonucleoside vanadyl complexes (Sigma-Aldrich, St. Louis, Mo.), 1× Complete Protease Inhibitor Cocktail, 28 mM β-mercaptoethanol, 120 U/ml RNasin Ribonuclease Inhibitor, and Zymolase (10 kU/ml)] for 30 min at 30° C. The spheroplasts were centrifuged for 4 min at 1300×g at 4° C. and washed twice in 0.1M potassium phosphate buffer, pH 7.4 containing 1.2M sorbitol. Spheroplasts were then resuspended in 70% ethanol and incubated for 1 hr at 4° C. Afterwards, cells were centrifuged at 1300×g at 4° C. for 4 min, washed with WASH buffer (0.3M sodium chloride, 30 mM sodium citrate, and 10% formamide), and incubated overnight at 30° C. in the dark with a hybridization mixture containing 0.3M sodium chloride, 30 mM sodium citrate, 10% dextran sulfate, 10% formamide, 2 mM ribonucleoside vanadyl complexes, and the TAMRA-labeled Stellaris probe mix for SUC2 (Biosearch Technologies, Novato, Calif.). After probe hybridization, labeled spheroplasts were centrifuged at 1300×g, the hybridization solution aspirated, and the spheroplasts incubated for 30 min at 30° C. in WASH buffer. Cells were then centrifuged and resuspended in a solution containing 0.3M sodium chloride and 30 mM sodium citrate. SUC2 mRNA co-localization with the ER was visualized using a DeltaVision imaging system (Applied Precision, Issaquah, Wash., USA). Images were processed by deconvolution.

### Computational Analyses

### SECReTE Score Calculation

Calculations of the SECReTE score were performed using the Perl programming language). For calculating motif number, the number of NNY repeats above a certain threshold was counted for three different positions (i.e. YNN, NYN, NNY, where Nis any nucleotide and Y is a pyrimidine).

### Gene Ontology

Definition of secretome was according to (Ast et al., 2013), this group includes all genes that contain TMD and/or signal sequence and are not mitochondrial. TMHMM tool was used to define TMD containing proteins. Cell wall and tail anchored genes were defined according to UniProt. Data from (Jan et al., 2014) was used to define other groups of genes and for defining human GO terms. The GO Slim Mapper tool (SGD) (worldwidewebdotyeastgenomedotorg/cgi-bin/GO/goSlimMapperdotpl) was used to classify ERTM10- and ERTM15-positive genes

### Permutation Test Analysis

For permutation analysis, each gene sequence was randomly shuffled 1000 times and the SECReTE was scored for each of the shuffled sequences. To evaluate the probability of SECReTE to appear randomly, a Z score was calculated for each gene according to the formula: Z=(Observed−mean)/STD. Observed is the value that was measured from the gene sequence. (e.g. SECReTE score for the gene). Mean is the average SECReTE score for all shuffled sequences of the gene. STD is the standard deviation of the SECReTE score from all shuffled sequences of the gene.

### Identification of Cell Wall Motif

Motif search was performed by MEME suits (Bailey et al., 2009), at memesuitedotorg/tools/meme.

### Results

### Identification of a Pyrimidine Repeat Motif in mRNAs Encoding Yeast Secretome Proteins

Because codons encoding hydrophobic residues are enriched in pyrimidines in their second position (Prilusky and Bibi, 2009), the present inventors examined mRNAs encoding secretome proteins in yeast for the presence of consecutive pyrimidine repeats every third nucleotide (i.e. YNN, NYN, or NNY) in the coding and UTR regions. First, we determined how many pyrimidine repeats might best differentiate secretome protein-encoding mRNAs from non-secretome protein-encoding mRNAs. For that, the number of repeats along an mRNA transcript was scored according to a defined threshold (e.g. 5, 7, 10, 12, and 15 repeats). To determine whether there is a correlation between gene length and presence of the repeats, the present inventors compared these two parameters (FIG. 1A). Based upon sequence analysis using the defined thresholds, they tentatively defined these repeats as “secretion-enhancing cis regulatory targeting elements” (SECReTE), termed: SECReTE5, 7, 10, 12, and 15. As shown (FIG. 1A), there is a direct correlation between SECReTE number and gene length for SECReTE5 and SECReTE7. However, the dependency on gene length is significantly weakened above SECReTE10 (FIG. 1A). This implies the presence of ≥10 consecutive repeats is not a random phenomenon and may be important.

If SECReTE repeats above 10 (e.g. SECReTE10) play a role in protein secretion, one may expect them to be more abundant in mRNAs encoding secretome proteins, as defined according to Ast et al. (Ast et al., 2013). To test this possibility, the present inventors divided the complete yeast genome into two groups: secretome and non-secretome, and calculated the fraction of transcripts that contain SECReTE in each group. They found transcripts coding for secretome proteins are enriched with SECReTE motifs >7 (FIG. 1B), as opposed to transcripts encoding non-secretome proteins. To test the number of repeats that give the most significant separation between secretome and non-secretome transcripts, the present inventors evaluated the different thresholds for their ability to classify mSMPs using receiver operator characteristics (ROC) analysis (Hanley and McNeil, 1982). Bona fide secretome protein-encoding transcripts were used as a true positive set and non-secretome protein-encoding transcripts were defined as true negatives. As seen (FIG. 1C), the SECReTE10 threshold maximally differentiated secretome transcripts from non-secretome transcripta. As SECReTE10 did not show a dependency upon gene length and gave the most significant separation between secretome and non-secretome transcripts, the present inventors used it as the threshold by which to define motif presence in subsequent analyses.

### SECReTE Abundance in mSMPs is Not Dependent on the Presence of a TMD

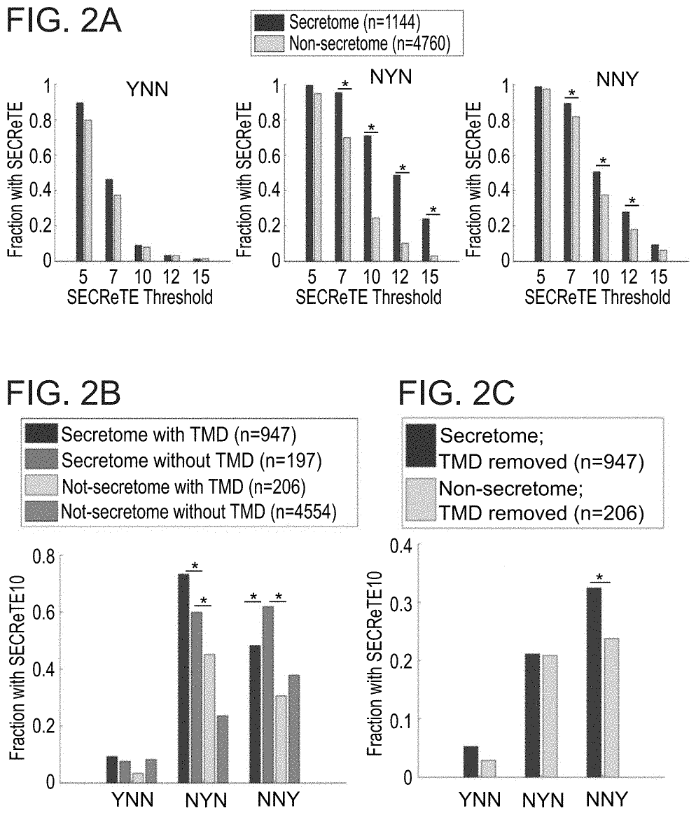

TMDs encoding mRNA sequences are enriched with uracil (U), mainly in the second position of the codon (NYN) (Wolfenden et al., 1979; Prilusky and Bibi, 2009). Since most secretome proteins contain TMDs, their presence alone might be the reason for motif enrichment in secretome transcripts. To ascertain whether SECReTE enrichment in mSMPs is not merely due to the presence of encoded TMDs, the present inventors determined at which position of the triplet the pyrimidine (Y) is located in the SECReTE10 elements: first (YNN); second (NYN); or third (NNY). They calculated SECReTE10 abundance separately for each position using only the coding sequences (i.e. from start codon to the stop codon) and without the UTRs. While the signal is present in the second position (FIG. 2A; NYN), as expected, it is also abundant in the third position of the codon (FIG. 2A; NNY). The latter finding implies that the TMD may not be the only factor that affects SECReTE enrichment in mSMPs. In contrast, the SECReTE10 element is poorly represented in the first position (FIG. 2A; YNN).

Next, they checked for the presence of SECReTE10 in mRNAs coding for TMD-containing proteins and soluble secreted proteins separately. As expected, more transcripts encoding TMD-containing secretome proteins contain SECReTE10 in the second position (NYN) than transcripts coding for soluble secreted proteins (FIG. 2B). However, the fraction of SECReTE10-containing transcripts coding for soluble secreted proteins in the third position (NNY) is even higher. This provides compelling evidence for SECReTE10 enrichment in transcripts independent of the encoded TMD regions. Correspondingly, when the TMD was artificially removed from the sequences of mRNAs encoding membrane proteins, the secretome genes were no longer enriched with second position SECReTE10s (FIG. 2C; NYN), although, the enrichment of SECReTE10 at the third position remained highly abundant (FIG. 2C; NNY).

### SECReTE Abundance is Not Dependent Upon Codon Composition

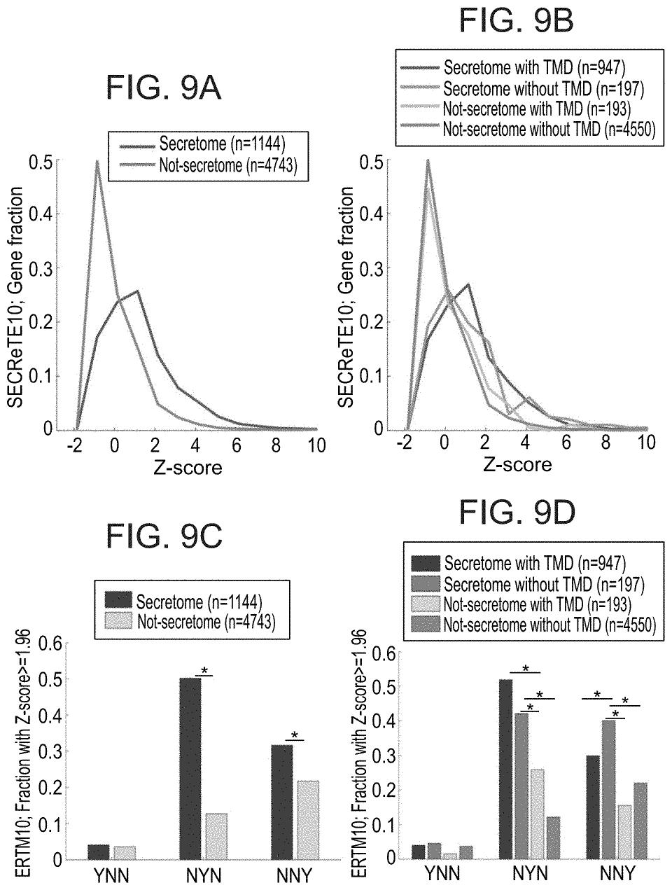

There is a possibility that SECReTE enrichment results from codon composition of the transcript. To check this possibility, the present inventors performed permutation test analysis. In this case, each gene sequence was randomly shuffled×1000, while codon composition remained constant. They then calculated the Z-score (i.e. number of standard deviations from the mean) of SECReTE10 for each gene to evaluate the probability of the signal to appear randomly. By looking at Z-score distribution in secretome and non-secretome genes, it can be concluded that SECReTE enrichment in mSMPs is not a random phenomenon and is not dependent on codon composition (FIG. 9A). This conclusion is valid for mSMPs encoding both membranal and soluble proteins (FIG. 9B). The present inventors also conducted the analysis for each codon position separately. For that, they calculated the fraction of genes with a significant Z-score (≥1.96) for each position separately. The fraction of genes with a significant Z-score was larger in secretome genes than in the non-secretome genes at both the second and third positions of the codon (FIG. 9C), strengthening the notion that SECReTE is significantly more enriched in those positions. This finding is not dependent on the presence of TMDs, since the fraction of genes with a significant Z-score was larger for both soluble and TMD-containing secretome transcripts, rather than for soluble and TMD-containing non-secretome transcripts (FIG. 9D).

### Gene Ontology (GO) Analysis

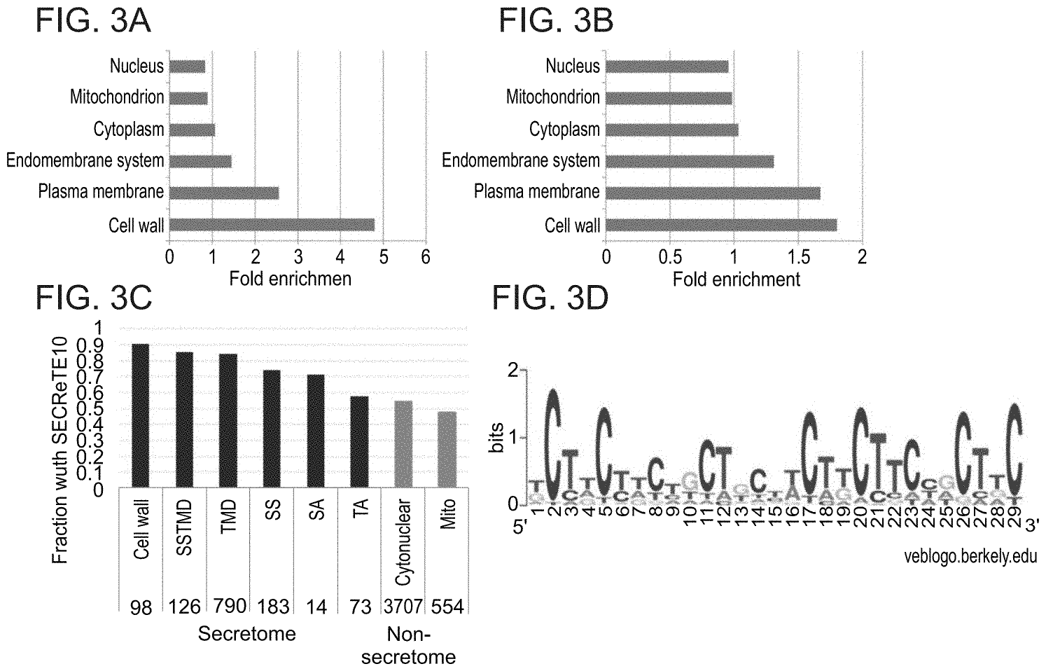

To determine those gene categories that are overrepresented in the population of SECReTE-containing genes, gene ontology (GO) enrichment analysis was conducted. When SECReTE10-positive genes were searched for GO enrichment (using all yeast genes as a background), unsurprisingly, membrane proteins were found to have a high enrichment score (fold enrichment=1.67) (FIG. 3A). The most SECReTE-enriched gene category was that comprising cell wall proteins (fold enrichment=1.8) (FIG. 3A). When 15 NNY repeats served as a threshold, the fold-change enrichment of the cell wall protein category increased to 4.8-fold (FIG. 3B). To further characterize the mRNAs enriched with SECReTE, the present inventors divided the secretome and non-secretome into subgroups and calculated the fraction of transcripts containing SECReTE10 in each category. In agreement with the GO analysis, more than 90% of mRNAs coding for cell wall proteins possess SECReTE10 elements and the cell wall proteins were the most SECReTE-rich (FIG. 3C). They found that 86% of mRNAs of proteins encoding both TMD and signal-sequence (SS) regions, as well as 84% of TMD-encoding secretome mRNAs, contain SECReTE10 (FIG. 3C). Of these, mRNAs encoding tail-anchored (TA) proteins contain the lowest number of transcripts with SECReTE10 in the secretome (FIG. 3C). TA proteins are known to translocate to the ER through an alternative pathway (GET) after being translated in the cytosol (Sharp and Li, 1987; Stefanovic and Hegde, 2007; Denic, 2012), and their transcripts are not enriched on ER membranes (Jan et al., 2014; Chartron et al., 2016). This could imply that SECReTE is more abundant in mRNAs undergoing translation on the ER. In contrast, transcript for non-secretome proteins (i.e. mitochondrial and cytonuclear) have the lowest abundance of SECReTE elements (FIG. 3C).

Since SECReTE is highly enriched in mRNAs coding for cell wall proteins, the present inventors wanted to know if it could be discovered using an unbiased motif search tool. For that, they analyzed the mRNA sequences of cell wall proteins using MEME to identify mRNA motifs. The most significant result obtained highly resembled the SECReTE10 repeat with either U or C (FIG. 3D). Importantly, they did not detect a protein motif within this mRNA motif, eliminating the possibility that the SECReTE element is dependent on the protein sequence.

### SECReTE Enrichment in mSMPs is Found in Both Prokaryotes and Higher Eukaryotes

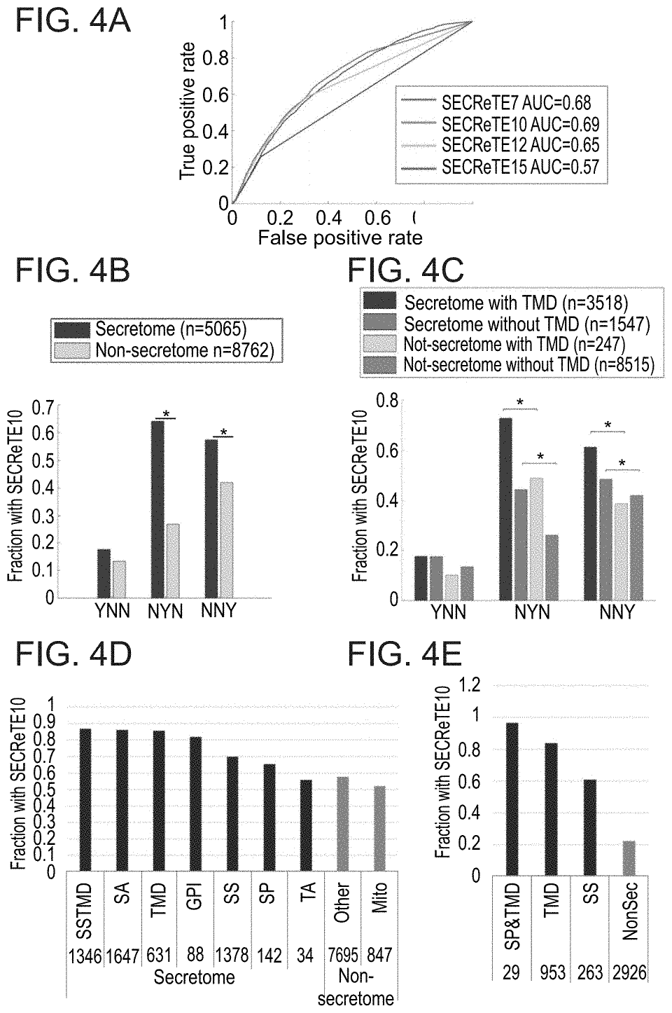

Either conservation or convergence in evolution are strong indications of significance. To check whether SECReTE enrichment in mSMPs is found in higher and lower organisms (e.g. humans and B. subtilis) the present inventors analyzed these genomes. In humans, as in S. cerevisiae, SECReTE10 gave the most significant separation between RNAs encoding secretome and non-secretome proteins, based on ROC analysis (FIG. 4A). After verifying that SECReTE10 does not correlate with gene length, 10 NNY repeats served as a threshold to define presence of the SECReTE motif. As in yeast, SECReTE is enriched in the second and third codon positions of secretome transcripts, in comparison to non-secretome transcripts (FIG. 4B). Also, a larger fraction of secretome transcripts that lack TMDs contain SECReTE, as compared to non-secretome transcripts either bearing or lacking TMDs (FIG. 4C). Interestingly, transcripts encoding GPI-anchored proteins, which are equivalent to cell wall proteins, were found to be highly enriched with SECReTE. In contrast, tail-anchored genes, as well as mitochondrial and cytonuclear genes, have a low SECReTE abundance as seen in yeast (FIG. 4D). A high abundance of SECReTE10 was detected in genes encoding secretome proteins from B. subtilis, in comparison to those encoding non-secretome proteins (FIG. 4E).

### Mutations in SECReTE Affect the Secretion of Endogenous Secretome Proteins

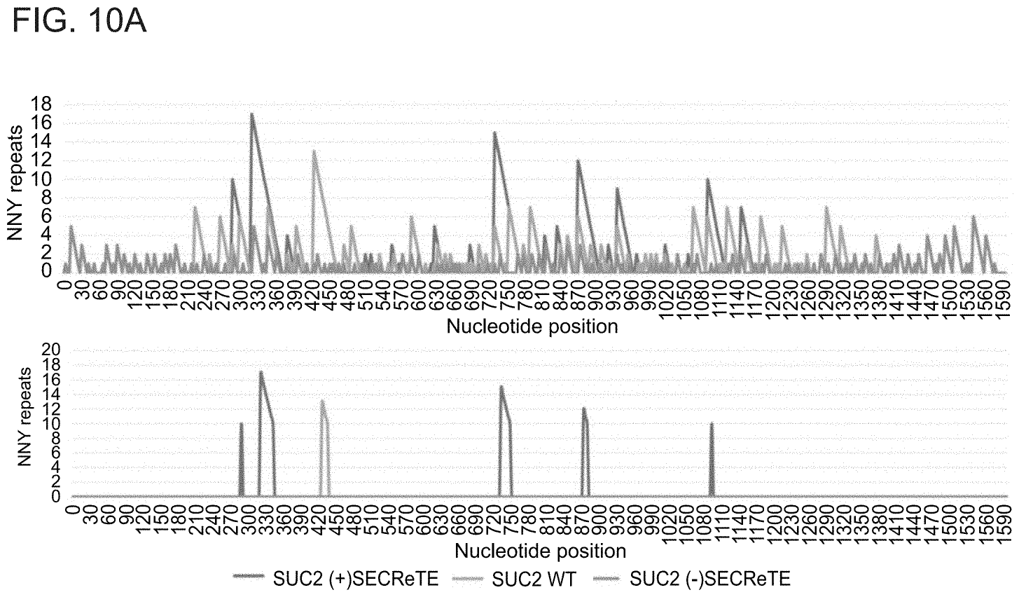

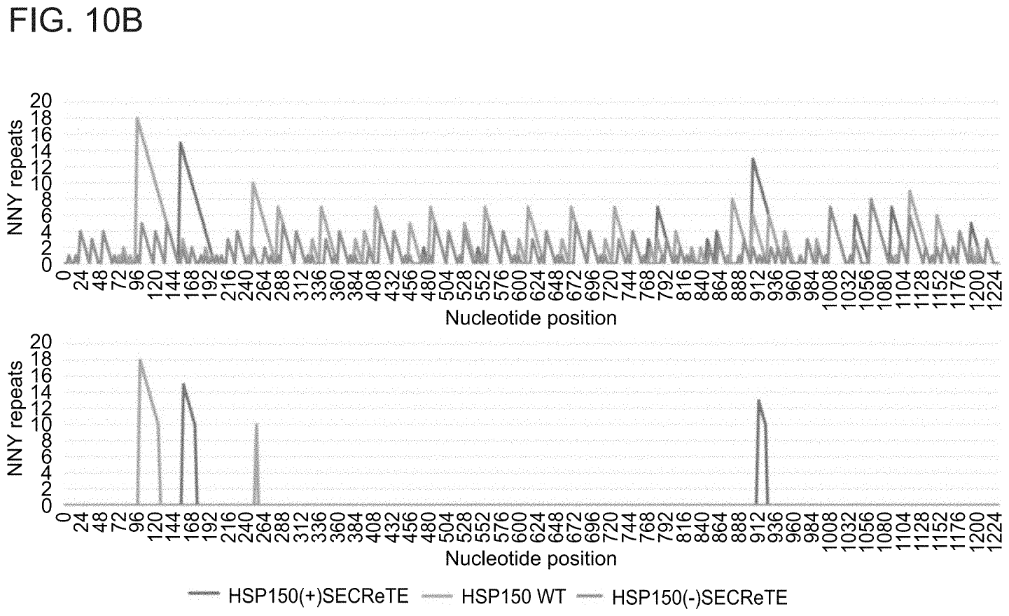

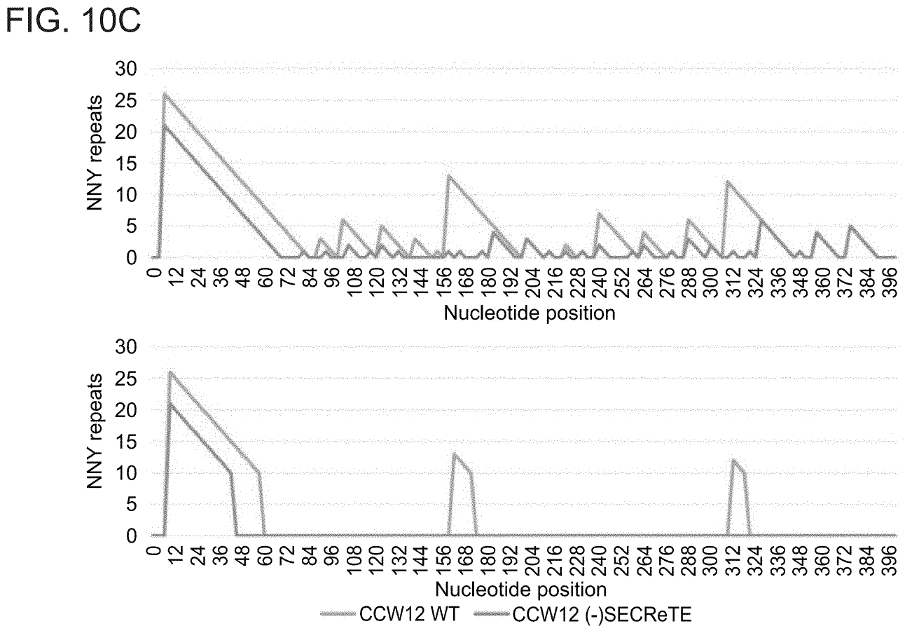

To further understand the significance of SECReTE and validate its importance to yeast cell physiology, the present inventors examined its relevance by elevating or decreasing the signal in selected genes. Three representative genes were chosen, based on their relatively short gene length, a detectable phenotype upon their deletion, and their function in different physiological pathways. These genes included: SUC2, which encodes a soluble secreted periplasmic enzyme; HSP150, which encodes a soluble media protein; and CCW12, which encodes a GPI-anchored cell wall protein. The overall SECReTE signal of the genes was increased by substituting any A or G found in the third codon position with a T or C, respectively, thereby enriching SECReTE presence along the entire gene [(+)SECReTE]. The reverse substitution, converting T to A or C to G, decreased the overall SECReTE signal [(−)SECReTE]. Crucially, these modifications were designed to ensure that only the SECReTE attribute of the mRNA sequence was altered, while no alterations in the encoded amino acid sequence were made. Furthermore, changes in the stability of the mRNA secondary structure were kept to within an acceptable range and the Codon Adaptation Index (CAI) remained within the optimal range of 0.8-1.0 (Sharp and Li, 1987). SECReTE mutations in SUC2, HSP150, and CCW12 are shown along the length of the gene, using a minimum threshold of either 1 NNY repeats or 10 NNY repeats, as shown in FIG. 10 (A-C; upper and lower parts respectively).

### SECReTE Mutations in SUC2 Alter Invertase Secretion

SUC2 codes for different forms of invertase translated from two distinct mRNAs, short and long, which differ only at their 5′ ends. While the longer mRNA codes for a secreted protein that contains a signal sequence, the signal sequence is omitted from the short isoform, which codes for a cytoplasmic protein. Secreted Suc2 expression is subjected to glucose repression; however, under inducing conditions (i.e., glucose depletion), Suc2 is trafficked through the secretory pathway to the periplasmic space of the cell. There, it catalyzes the hydrolysis of sucrose to glucose and fructose, this enzymatic activity being responsible for the ability of yeast to utilize sucrose as a carbon source, and can be measured by a biochemical assay (i.e. invertase activity), both inside and outside of the cell. The effect of SECReTE mutations on Suc2 function was tested by examining the ability of mutants to grow on sucrose-containing media by drop-test. Interestingly, the growth rate of SUC2(−)SECReTE on sucrose plates was decreased, while the SUC2(+)SECReTE mutant exhibited better growth in comparison to WT cells (FIG. 5A), even though no growth change was detected on YPD plates. These findings suggest that SECReTE strength affects the secretion of Suc2. These changes in Suc2 secretion could result from changes in SUC2 transcription, Suc2 production, and/or altered rates of secretion. To distinguish between possibilities, WT cells, suc2A, and SUC2 SECReTE mutants were subjected to invertase assays. The invertase assay enables the quantification of secreted Suc2, as well as internal Suc2, by calculating the amount of glucose produced from sucrose. As expected, under glucose repressing conditions (e.g. 2% glucose) the levels of both secreted and internal Suc2 were very low. When cells were grown on media containing low glucose (e.g. 0.05% glucose) to promote the expression of the secreted enzyme, secreted Suc2 levels were altered due to changes in SECReTE. Corresponding to the drop-test results, a significant decrease in secreted invertase was detected with SUC2(−)SECReTE cells, while a significant increase was detected with Suc2(+)SECReTE cells, in comparison to WT cells. No Suc2 secretion was detected from suc2Δ cells (FIG. 5B, secreted). If SECReTE mutations affect the efficiency of Suc2 secretion, but not its synthesis, then Suc2 accumulation would be expected to occur in SUC2(−)SECReTE cells. Likewise, as a decrease of internal invertase would be expected to occur in SUC2(+)SECReTE cells. However, this was not the case as the internal amount of Suc2 was decreased in SUC2(−)SECReTE cells and was slightly increased in Suc2(+)SECReTE cells (FIG. 5B, internal). These findings suggest that SECReTE alterations in SUC2 likely affect protein production.

### SECReTE Mutations alter Hsp150 Secretion and Cell Wall Stability

Next, the present inventors wanted to study the importance of SECReTE in HSP150. Hsp150 is a component of the outer cell wall and while the exact function of Hsp150 is unknown, it is required for cell wall stability and resistance to cell wall-perturbing agents, such as Calcofluor White (CFW) and Congo Red (CR). While hsp150Δ cells are more sensitive to cell wall stress, the overproduction of Hsp150 increases cell wall integrity (Hsu et al., 2015). Hsp150 is secreted efficiently into the growth media and its expression is increased upon heat shock (Russo et al., 1992, 1993)). The effect of modifying SECReTE in HSP150 was examined via drop-test by testing the sensitivity of HSP150(−)SECReTE and HSP150(+)SECReTE cells to added CFW, in comparison to WT and hsp150Δ cells. As can be seen from FIG. 5C, while the HSP150(−) SECReTE strain was more sensitive to CFW as compared to WT cells, the HSP150(+)SECReTE strain was more resistant to CFW. As expected, hsp150Δ cells are the most susceptible to CFW (FIG. 5C). HSP150 strains were also subjected to Western blot analysis to measure levels of the mutant proteins. Since HSP150 secretion is elevated upon heat-shock (Russo et al., 1992, 1993), cells were grown at 37° C. before protein extraction. Protein was extracted from both the growth medium and cells to detect both external and internal protein levels, respectively. The amount of Hsp150 secreted to the medium was decreased in HSP150(−)SECReTE cells and elevated in HSP150(+)SECReTE cells, in comparison to WT cells (FIG. 5D). Similar to Suc2, the internal amount of Hsp150 was also decreased in HSP150(−)SECReTE cells, as compared to WT cells (FIG. 5D). This could mean that secretion per se was not significantly attenuated by the reduction in SECReTE strength. As the internal amount of Hsp150 in HSP150(+)SECReTE cells was similar to that of WT cells, it may be concluded that SECReTE alteration in HSP150 also likely affects protein production.

### SECReTE Mutations in CCW12 Alter Cell Wall Stability

CCW12 encodes a glycophosphatidylinositol (GPI)-anchored cell wall protein that localizes to regions of the newly synthesized cell wall and maintains wall stability during bud emergence and shmoo formation. Deletion of CCW12 was shown to cause hypersensitivity to cell wall destabilizing agents, like hygromycin B (HB) (Ragni et al., 2007, 2011). Since the SECReTE score is very high in CCW12, it was not possible to further increase the signal. Therefore, the present inventors generated only CCW12(−)SECReTE cells and tested their ability to grow on HB-containing plates. As seen with HSP150(−)SECReTE (FIG. 5C), it was found that the CCW12(−) SECReTE mutation rendered cells sensitive to cell wall perturbation, in comparison to WT cells (FIG. 5E).

### SECReTE Addition Affects Secretion of an Exogenous Naïve Protein

The ability of SECReTE addition to improve the secretion of an exogenous protein would not only be substantial evidence for its importance, but also could be a useful, low-cost, industrial tool to improve the secretion of recombinant proteins without changing protein sequence. To test that, the present inventors employed a GFP transcript construct bearing the encoded SS of Gas1 (SSGas1) at the 5′ end. SSGasI enables the secretion of GFP protein to the medium, although its secretion was not as efficient in comparison to other SS-fused GFP proteins, such as SSKar2 (FIG. 5F). To potentially improve the secretion of SSGas1, the present inventors added an altered 3′UTR sequence of Gas1 that contained SECReTE [i.e. in which all A's and G's were replaced with T's and C's, respectively; SSGasI-3′UTRGASI(+)SECReTE]. They then tested the effect of SECReTE addition upon secretion of GFP into the media. They found that the addition of SECReTE to the 3′UTR of GasI-GFP improved the secretion of GFP secretion into the media, in comparison to SSGasI-GFP, and was similar to that of SSKar2-GFP (FIG. 5F).

### The Effect of SECReTE Mutations on mRNA Levels

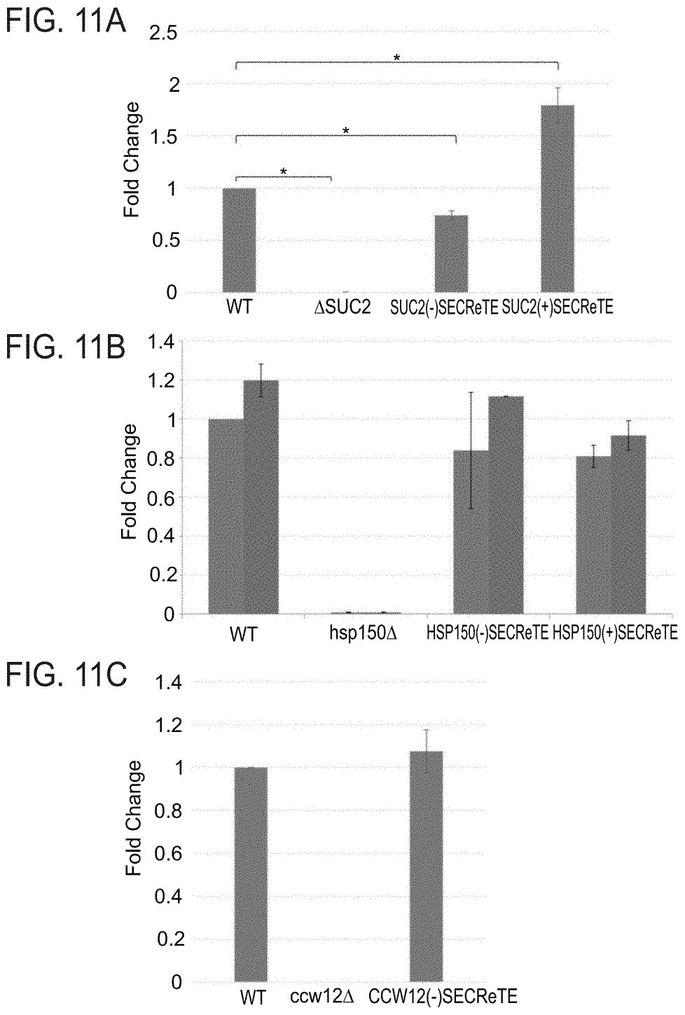

As protein levels may be altered by (−)SECReTE and (+)SECReTE mutations (FIG. 5B, D, and F), the present inventors examined whether changes in gene transcription or mRNA stability are involved. Quantitative real-time (qRT) PCR was employed to check whether mRNA levels of SUC2, HSP150, and CCW12 are affected by SECReTE strength. It was found that SUC2(−)SECReTE mRNA levels were almost 30% lower than in SUC2 WT cells, while SUC2(+)ERTM levels were ˜50% higher than WT (FIG. 11A). This change in mRNA levels might be the cause for the ability of SUC2(+)SECReTE mutant to increase protein production and, therefore, grow better on sucrose-containing medium (FIG. 5A,B).

The effect of SECReTE mutation on HSP150 mRNA levels was also studied. Interestingly, it was found that the mRNA level of HSP150(−)SECReTE was similar to WT, while that of HSP150(+)SECReTE was slightly decreased (FIG. 11B). Thus, the change in Hsp150 protein levels and sensitivity to CFW due to SECReTE alteration (FIG. 5C and D) is not explained by changes in mRNA levels. Likewise, SECReTE mutations in CCW12(−)SECReTE did not cause a significant change in its mRNA level (FIG. 11C). Therefore, the increased sensitivity of CCW12(−)SECReTE to HB (FIG. 5E) is not due to a decrease in CCW12 mRNA.

### The Effect of SECReTE Mutation on SUC2 mRNA Localization

To test whether SECReTE has a role in dictating mRNA localization, the present inventors visualized SUC2 mRNA by single-molecule FISH (smFISH) using specific fluorescent probes and tested the influence of SECReTE alteration on the level of SUC2 mRNA co-localization with ER. They used Sec63-GFP as an ER marker and calculated the percentage of granules per cell that co-localized either with or not with the ER, or were adjacent to the ER. The level of co-localization between SUC2(−)SECReTE mRNA granules and Sec63-GFP was found to decrease slightly in comparison to WT SUC2 mRNA granules (FIGS. 6A and B). In contrast, w a significant increase of ˜50% was observed in the level of co-localization of SUC2(+)SECReTE mRNA granules with the ER, in comparison to WT SUC2 mRNA (FIG. 6A and B). These findings suggest that SECReTE has role in the targeting of SUC2 mRNA to the ER.

### Identification of Potential SECReTE-Binding Proteins

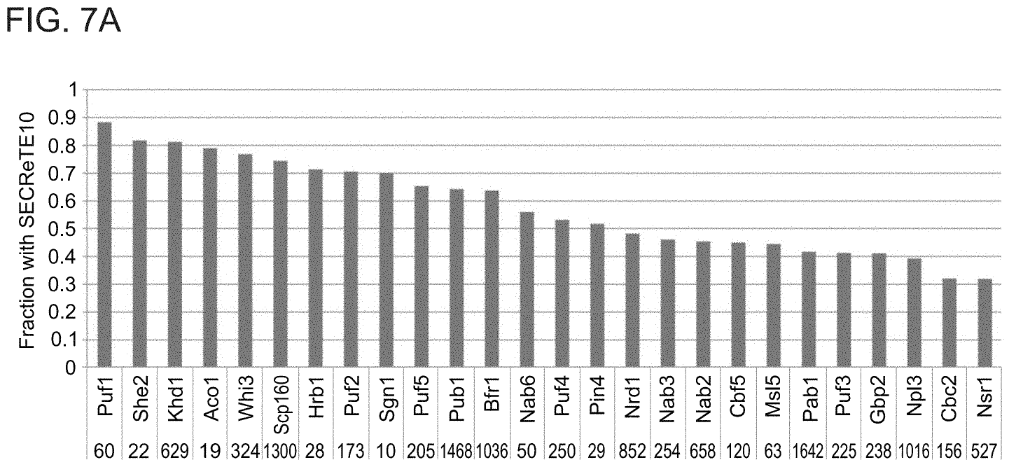

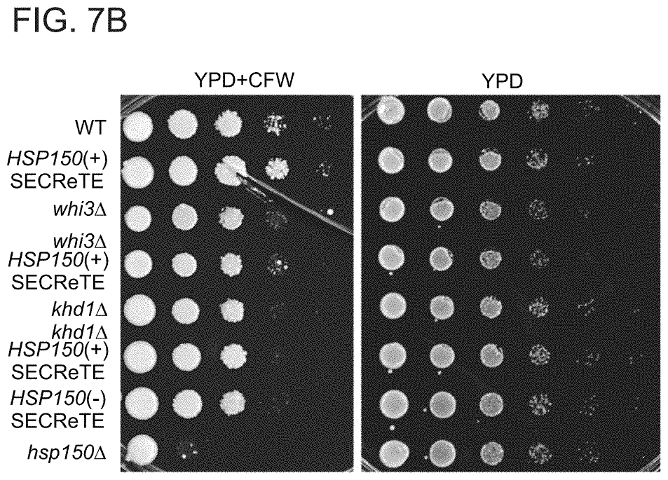

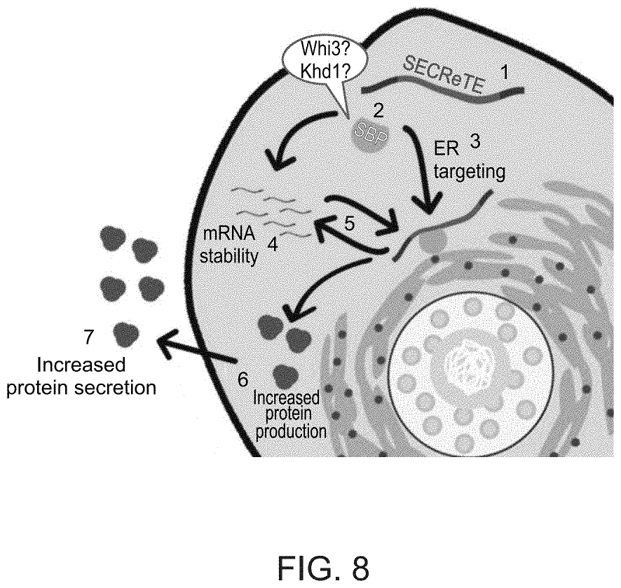

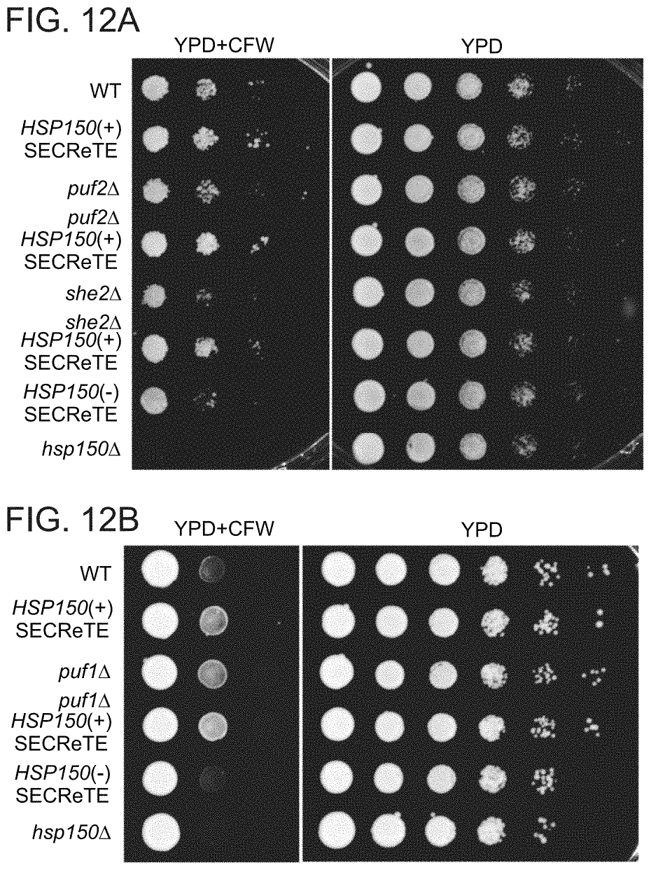

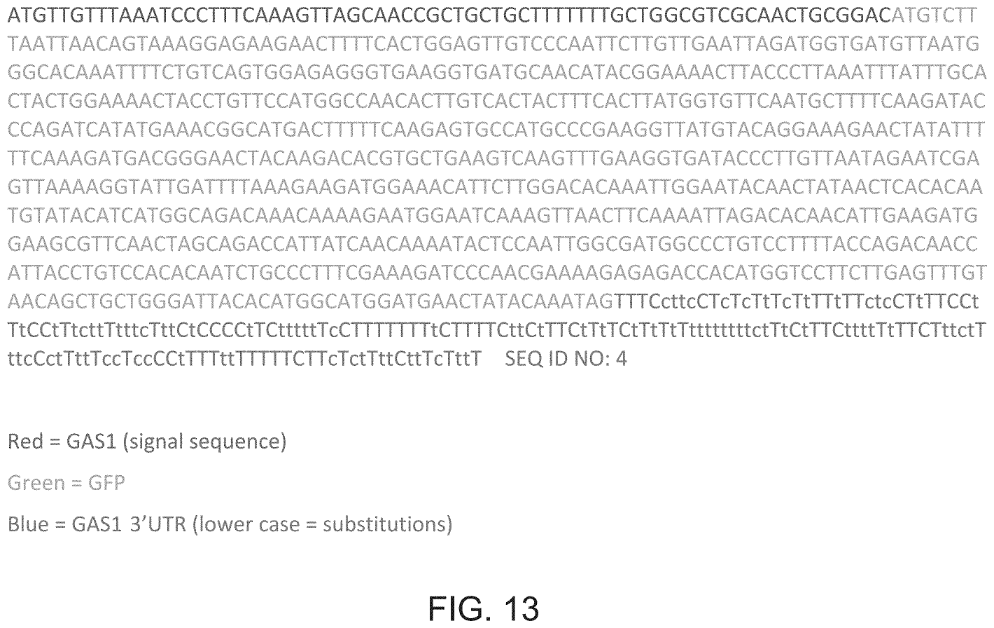

To further elucidate the role of SECReTE it is essential to identify its binding partners, presumably RBPs. Large-scale approaches were previously used to identify mRNAs that are bound >40 known RBPs in yeast (Colomina et al., 2008; Hasegawa et al., 2008; Hogan et al., 2008). To obtain a list of potential SECReTE-binding proteins (SBPs) the present inventors searched the datasets for RBPs that bind mRNAs highly enriched with SECReTE. For each RBP, they calculated what fraction of its bound transcripts that contain SECReTE10. RBPs found to bind large fractions of SECReTE10-containing mRNAs included Bfr1, Whi3, Puf1, Puf2, Scp160, and Khd1 (FIG. 7A), and were all previously shown to bind mSMPs. To test which of candidates bind SECReTE, each of the genes these RBPs was deleted in either WT or HSP150(+)SECReTE cells. They hypothesized that the deletion of a genuine SBP might confer hypersensitivity to CFW and eliminate the growth rate differences between WT and HSP150(+)SECReTE cells observed on CFW-containing plates (FIG. 5C). When PUF1, PUF2, or SHE2 were deleted it was found that HSP150(+)SECReTE strain was still more resistant to CFW than WT cells (FIGS. 12A-B). One possible explanation for this lack of effect is that these RBPs either do not bind HSP150 or that they are redundant with other SBPs. However, it was found that the deletion of either WHI3 or KHD1 eliminated the differences between WT and HSP150(+)SECReTE strains on CFW-containing plates (FIG. 7B). This suggests Whi3 and Khd1 bind HSP150 mRNA and possibly other secretome mRNAs, and even WT cells alone were rendered more sensitive to CFW in their absence (FIG. 7B).

### REFERENCES

Aronov, S., Gelin-Licht, R., Zipor, G., Haim, L., Safran, E., and Gerst, J. E. (2007). mRNAs Encoding Polarity and Exocytosis Factors Are Cotransported with the Cortical Endoplasmic Reticulum to the Incipient Bud in Saccharomyces cerevisiae. Mol. Cell. Biol. 27, 3441-3455.

Ast, T., Cohen, G., and Schuldiner, M. (2013). A network of cytosolic factors targets SRP-independent proteins to the endoplasmic reticulum. Cell 152, 1134-1145.

Bailey, T. L., Boden, M., Buske, F. A., Frith, M., Grant, C. E., Clementi, L., Ren, J., Li, W. W., and Noble, W. S. (2009). MEME SUITE: tools for motif discovery and searching. Nucleic Acids Res. 37, W202-W208.

Blobel, G., and Dofferstein, B. (1975). Transfer of proteins across membranes. I. Presence of proteolytically processed and unprocessed nascent immunoglobulin light chains on membrane bound ribosomes of murine myeloma. J. Cell Biol. 67, 835-851.

Buxbaum, A. R., Haimovich, G., and Singer, R. H. (2015). In the right place at the right time: visualizing and understanding mRNA localization. Nat. Rev. Mol. Cell Biol. 16, 95-109.

Cal, Y., Futcher, B., Waern, K., Shou, C., and Raha, D. (2013). Effects of the Yeast RNA-Binding Protein Whi3 on the Half-Life and Abundance of CLN3 mRNA and Other Targets. PLoS One 8, e84630.

Chartron, J. W., Hunt, K. C. L., and Frydman, J. (2016). Cotranslational signal-independent SRP preloading during membrane targeting. Nature 536, 224-228.

Chen, Q., Jagannathan, S., Reid, D. W., Zheng, T., and Nicchitta, C. V (2011). Hierarchical regulation of mRNA partitioning between the cytoplasm and the endoplasmic reticulum of mammalian cells. Mol. Biol. Cell 22, 2646-2658.

Chin, A., and Lécuyer, E. (2017). RNA localization: Making its way to the center stage. Biochim. Biophys. Acta 1861, 2956-2970.

Christiansen, T., Foy, B. D., Wall, L., and Orwant, J. (2012) Programming Perl: Unmatched power for text processing and scripting. O'Reilly Media Inc. ISBN:0596004923 9780596004927

Colomina, N., Ferrezuelo, F., Wang, H., Aldea, M., and Garí, E. (2008). Whi3, a developmental regulator of budding yeast, binds a large set of mRNAs functionally related to the endoplasmic reticulum. J. Biol. Chem. 283, 28670-28679.

Cui, X. a., and Palazzo, A. F. (2014). Localization of mRNAs to the endoplasmic reticulum. Wiley Interdiscip. Rev. RNA 5, 481-492.

Cui, X. A., Zhang, H., Palazzo, A. F., Fugate, R. D., and Reichlin, M. (2012). p180 Promotes the Ribosome-Independent Localization of a Subset of mRNA to the Endoplasmic Reticulum. PLoS Biol. 10, e1001336.

Denic, V. (2012). A portrait of the GET pathway as a surprisingly complicated young man. Trends Biochem. Sci. 37, 411-417.

Diehn, M., Eisen, M. B., Botstein, D., and Brown, P. O. (2000). Large-scale identification of secreted and membrane-associated gene products using DNA microarrays. Nat. Genet. 25, 58-62.

Gerst, J. E. (2008). Message on the web: mRNA and ER co-trafficking. Trends Cell Biol. 18, 68-76.

Gilmore, R., Walter, P., and Blobel, G. (1982). Protein translocation across the endoplasmic reticulum. II. Isolation and characterization of the signal recognition particle receptor. J. Cell Biol. 95, 470-477.

Goldstein, A., and Lampen, J. O. (1975). Beta-D-fructofuranoside fructohydrolase from yeast. Methods Enzymol. 42, 504-511.

Haim, L. and Gerst, J. E. (2009) m-TAG: A PCR-based genomic integration method to visualize the localization of specific endogenous mRNAs in vivo in yeast. Nat. Protocols 4, 1274-1284.

Hamilton, R. S., and Davis, I. (2011). Identifying and searching for conserved RNA localisation signals. Methods Mol. Biol. 714, 447-466.

Hanley, J. A. and McNeil, B. J. (1982) The meaning and use of the area under a receiver operating characteristic. Radiology 143, 29-36.

Hasegawa, Y., Irie, K., and Gerber, A. P. (2008). Distinct roles for Khdlp in the localization and expression of bud-localized mRNAs in yeast. RNA 14, 2333-2347.

Hogan, D. J., Riordan, D. P., Gerber, A. P., Herschlag, D., and Brown, P. O. (2008). Diverse RNA-binding proteins interact with functionally related sets of RNAs, suggesting an extensive regulatory system. PLoS Biol. 6, 2297-2313.

Houshmandi, S. S., and Olivas, W. M. (2005). Yeast Puf3 mutants reveal the complexity of Puf-RNA binding and identify a loop required for regulation of mRNA decay. RNA 11, 1655-1666.

Hsu, P.-H., Chiang, P.-C., Liu, C.-H., and Chang, Y.-W. (2015). Characterization of Cell Wall Proteins in Saccharomyces cerevisiae Clinical Isolates Elucidates Hsp150p in Virulence. PLoS One 10, e0135174.

Irie, K., Tadauchi, T., Takizawa, P. A., Vale, R. D., Matsumoto, K., and Herskowitz, I. (2002). The Khd1 protein, which has three KH RNA-binding motifs, is required for proper localization of ASH1 mRNA in yeast. EMBO J. 21, 1158-1167.

Ito, W., Li, X., Irie, K., Mizuno, T., and Irie, K. (2011). RNA-Binding Protein Khd1 and Ccr4 Deadenylase Play Overlapping Roles in the Cell Wall Integrity Pathway in Saccharomyces cerevisiae Eukaryot. Cell 10, 1340-1347.

Jagannathan, S., Reid, D. W., Cox, A. H., Jagannathan, S., Reid, D. W., Cox, A. H., and Nicchitta, C. V (2014). De novo translation initiation on membrane-bound ribosomes as a mechanism for localization of cytosolic protein mRNAs to the endoplasmic reticulum. RNA 20, 1489-1498.

Jan, C. H., Williams, C. C., and Weissman, J. S. (2014). Principles of ER cotranslational translocation revealed by proximity-specific ribosome profiling. Science 346, 1257521.

Jan, C. H., Williams, C. C., and Weissman, J. S. (2015). Response to Comment on “Principles of ER cotranslational translocation revealed by proximity-specific ribosome profiling.” Science (80-.). 348.

Johnson, N., Powis, K., and High, S. (2013). Post-translational translocation into the endoplasmic reticulum. Biochim. Biophys. Acta-Mol. Cell Res. 1833, 2403-2409.

Kejiou, N. S., and Palazzo, A. F. (2017). mRNA localization as a rheostat to regulate subcellular gene expression. Wiley Interdiscip. Rev. RNA 8, e1416.

Kraut-Cohen, J., and Gerst, J. E. (2010). Addressing mRNAs to the ER: cis sequences act up! Trends Biochem. Sci. 35, 459-469.

Kraut-Cohen, J., Afanasieva, E., Haim-Vilmovsky, L., Slobodin, B., Yosef, I., Bibi, E., and Gerst, J. E. (2013). Translation- and SRP-independent mRNA targeting to the endoplasmic reticulum in the yeast Saccharomyces cerevisiae. Mol. Biol. Cell 24, 3069-3084.

Lerner, R. S., Seiser, R. M., Zheng, T., Lager, P. J., Reedy, M. C., Keene, J. D., and Nicchitta, C. V (2003). Partitioning and translation of mRNAs encoding soluble proteins on membrane-bound ribosomes. Rna 9, 1123-1137.

Martin, K. C., and Ephrussi, A. (2009). mRNA localization: gene expression in the spatial dimension. Cell 136, 719-730.

Mutka, S. C., and Walter, P. (2001). Multifaceted Physiological Response Allows Yeast to Adapt to the Loss of the Signal Recognition Particle-dependent Protein-targeting Pathway. Mol. Biol. Cell 12, 577-588.

Novick, P., and Schekman, R. (1979). Secretion and cell-surface growth are blocked in a temperature-sensitive mutant of Saccharomyces cerevisiae. Proc. Natl. Acad. Sci. U.S.A. 76, 1858-1862.

Olivas, W., and Parker, R. (2000). The Puf3 protein is a transcript-specific regulator of Gen. Genet. 239, 273-280.

Saint-Georges, Y., Garcia, M., Delaveau, T., Jourdren, L., Le Crom, S., Lemoine, S., Tanty, V., Devaux, F., and Jacq, C. (2008). Yeast Mitochondrial Biogenesis: A Role for the PUF RNA-Binding Protein Puf3p in mRNA Localization. PLoS One 3, e2293.

Saraogi, I., Shan, S., and Ishu Saraogi, S. (2011). Molecular mechanism of co-translational protein targeting by the signal recognition particle. Traffic 12, 535-542.

Schmid, M., Jaedicke, A., Du, T.-G., and Jansen, R.-P. (2006). Coordination of Endoplasmic Reticulum and mRNA Localization to the Yeast Bud. Curr. Biol. 16, 1538-1543.

Schwartz, T. U. (2007). Origins and evolution of cotranslational transport to the ER. Adv. Exp. Med. Biol. 607, 52-60.

Shahbabian, K., and Chartrand, P. (2012). Control of cytoplasmic mRNA localization. Cell. Mol. Life Sci. 69, 535-552.

Sharp, P. M., and Li, W. H. (1987). The codon Adaptation Index—a measure of directional synonymous codon usage bias, and its potential applications. Nucleic Acids Res. 15, 1281-1295.

Stefanovic, S., and Hegde, R. S. (2007). Identification of a Targeting Factor for Posttranslational Membrane Protein Insertion into the ER. Cell 128, 1147-1159.

Tang, H., Song, M., He, Y., Wang, J., Wang, S., Shen, Y., Hou, J., and Bao, X. (2017). Engineering vesicle trafficking improves the extracellular activity and surface display efficiency of cellulases in Saccharomyces cerevisiae. Biotechnol. Biofuels 10, 53.

Troy A A, H. (2014). A Simplified Method for Measuring Secreted Invertase Activity in Saccharomyces cerevisiae. Biochem. Pharmacol. Open Access 3.

Verges, E., Colomina, N., Gari, E., Gallego, C., and Aldea, M. (2007). Cyclin Cln3 Is Retained at the ER and Released by the J Chaperone Ydj1 in Late G1 to Trigger Cell Cycle Entry. Mol. Cell 26, 649-662.

Walter, P., and Blobel, G. (1981). Translocation of proteins across membranes III. Signal recognition protein (SRP) causes signal sequence-dependent and site specific arrest of chain elongation that is released by microsomal membranes. J. Cell Biol. 91, 557-561.

Weis, B. L., Schleiff, E., and Zerges, W. (2013). Protein targeting to subcellular organelles via mRNA localization. Biochim. Biophys. Acta-Mol. Cell Res. 1833, 260-273.

Wolfenden, R. V, Cullis, P. M., and Southgate, C. C. (1979). Water, protein folding, and the genetic code. Science 206, 575-577.

Ye, J., Coulouris, G., Zaretskaya, I., Cutcutache, I., Rozen, S., and Madden, T. L. (2012). Primer-BLAST: A tool to design target-specific primers for polymerase chain reaction. BMC Bioinformatics 13, 134.

Zhang, T., Lei, J., Yang, H., Xu, K., Wang, R., and Zhang, Z. (2011). An improved method for whole protein extraction from yeast Saccharomyces cerevisiae. Yeast 28, 795-798.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. An isolated polynucleotide comprising a transcriptional unit, wherein the transcriptional unit comprises:
(i) a nucleic acid sequence that encodes a secreted protein of interest;
(ii) an endoplasmic reticulum (ER) targeting sequence as set forth in SEQ ID NO: 2, said ER targeting sequence being heterologous to said secreted protein of interest;
(iii) a promoter; and
(iv) a transcription termination site, wherein said nucleic acid sequence that encodes a protein of interest and said ER targeting sequence are positioned between said promoter and said transcription termination site;
wherein when said ER targeting sequence is comprised in said nucleic acid sequence that encodes said protein of interest, said nucleic acid sequence has been codon optimized to comprise said ER targeting sequence.

2. The isolated polynucleotide of claim 1, wherein said ER targeting sequence does not comprise nucleotides that encode for said secreted protein of interest.

3. The isolated polynucleotide of claim 1, wherein said ER targeting sequence comprises at least 15 consecutive repeats of the sequence NNY, wherein N is any base and Y is a pyrimidine.

4. The isolated polynucleotide of claim 1, wherein said ER targeting sequence does not comprise more than 10 consecutive thymines.

5. The isolated polynucleotide of claim 1, wherein said transcriptional unit further encodes a signal peptide sequence.

6. The isolated polynucleotide of claim 5, wherein said signal peptide sequence is heterologous to said protein of interest.

7. The isolated polynucleotide of claim 1, wherein said protein of interest is a human protein.

8. An RNA transcribed from the polynucleotide of claim 1.

9. A cell comprising the isolated polynucleotide of claim 1.

10. The cell of claim 9, wherein said cell is of a species selected from the group consisting of a bacterial species, a fungal species, a plant species, an insect species and a mammalian species.

11. An expression construct comprising a nucleic acid sequence as set forth in SEQ ID NO: 2 and a cloning site, wherein a position of said cloning site is selected such that upon insertion of a sequence which encodes a protein of interest into said cloning site, following expression in a cell, an mRNA is transcribed which encodes said protein of interest and further comprises a transcription product of said nucleic acid sequence, wherein said SEQ ID NO: 2 is not comprised in a sequence that encodes for a protein.

12. The expression construct of claim 11, further comprising a promoter suitable for expressing said protein of interest in a cell.

13. A method of expressing a protein in a cell, the method comprising introducing into the cell the isolated polynucleotide of claim 1, thereby expressing the protein.

14. A method of generating a protein comprising expressing the protein according to any one of claim 13 and isolating the protein, thereby generating the protein.


SEQUENCE LISTING (6 sequences)

SEQ ID NO: 1 - DNA, 21 bp, Artificial Sequence
  Endoplasmic reticulum (ER) targeting sequence
  misc_feature (1)..(2): n is a, c, g, or t
  misc_feature (4)..(5): n is a, c, g, or t
  misc_feature (7)..(8): n is a, c, g, or t
  misc_feature (10)..(11): n is a, c, g, or t
  misc_feature (13)..(14): n is a, c, g, or t
  misc_feature (16)..(17): n is a, c, g, or t
  misc_feature (19)..(20): n is a, c, g, or t
nnynnynnynnynnynnynny

SEQ ID NO: 2 - DNA, 30 bp, Artificial Sequence
  Endoplasmic reticulum (ER) targeting sequence
  misc_feature (1)..(2): n is a, c, g, or t
  misc_feature (4)..(5): n is a, c, g, or t
  misc_feature (7)..(8): n is a, c, g, or t
  misc_feature (10)..(11): n is a, c, g, or t
  misc_feature (13)..(14): n is a, c, g, or t
  misc_feature (16)..(17): n is a, c, g, or t
  misc_feature (19)..(20): n is a, c, g, or t
  misc_feature (22)..(23): n is a, c, g, or t
  misc_feature (25)..(26): n is a, c, g, or t
  misc_feature (28)..(29): n is a, c, g, or t
nnynnynnynnynnynnynnynnynnynny

SEQ ID NO: 3 - DNA, 69 bp, Artificial Sequence
  GAS1 (signal sequence)
atgttgtttaaatccctttcaaagttagcaaccgctgctgctttttttgctggcgtcgcaactgcggac

SEQ ID NO: 4 - DNA, 979 bp, Artificial Sequence
  exemplary sequence that may be used for expression of GFP
atgttgtttaaatccctttcaaagttagcaaccgctgctgctttttttgctggcgtcgcaactgcggacatgtctttaattaacagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcacttatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatagtttccttcctctctttctttttttctccttttcctttcctttcttttttctttctccccttcttttttccttttttttcttttcttctttcttttcttttttttttttttctttctttcttttttttctttcttttccctttttcctcccctttttttttttcttctcttttctttctttt

SEQ ID NO: 5 - protein, 4 aa, Artificial Sequence
  peptide  amino acid sequence
KDEL

SEQ ID NO: 6 - DNA, 30 bp, Artificial Sequence
  a polynucleotide nucleic acid sequence
agctacacccaccacctcatctacctctac